(19) United States
(12) Patent Application Publication
(10) Pub. No.: US 2025/0210141 A1
(43) Pub. Date: Jun. 26, 2025

(54) ENHANCED MAPPING AND ALIGNMENT OF NUCLEOTIDE READS UTILIZING AN IMPROVED HAPLOTYPE DATA STRUCTURE WITH ALLELE-VARIANT DIFFERENCES

(71) Applicant: Illumina, Inc., San Diego, CA (US)

(72) Inventor: Michael Ruehle, Fort Worth, TX (US)

(21) Appl. No.: 18/990,380

(22) Filed: Dec. 20, 2024

Related U.S. Application Data
(60) Provisional application No. 63/613,574, filed on Dec. 21, 2023.

(51) Int. Cl. G16B 30/10

(52) U.S. Cl. CPC G16B 30/10

(57) ABSTRACT

This disclosure describes methods, non-transitory computer readable media, and systems that implement improved mapping and alignment of nucleotide reads with genomic regions of a reference genome. For instance, the disclosed systems can identify, for one or more candidate alignments between nucleotide reads from a genomic sample with a primary contiguous sequence at respective genomic regions of a reference genome, allele-variant differences between the primary contiguous sequence and population haplotypes within the respective genomic regions to generate alignment score adjustments for each population haplotype. To facilitate the disclosed methods for improved mapping and alignment of nucleotide reads, the disclosed systems can utilize a haplotype data structure comprising a hierarchical partitioning of a reference genome into reference bins representing respective genomic regions and encoding region-specific allele-variant differences between population haplotypes and a primary contiguous sequence of the reference genome.

### CROSS-REFERENCE TO RELATED APPLICATIONS

This application claims priority to and the benefit of U.S. Provisional Patent Application No. 63/613,574, entitled, “ENHANCED MAPPING AND ALIGNMENT OF NUCLEOTIDE READS UTILIZING AN IMPROVED HAPLOTYPE DATA STRUCTURE WITH ALLELE-VARIANT DIFFERENCES,” filed on Dec. 21, 2023 (IP-2590-PRV). The aforementioned application is hereby incorporated by reference in its entirety.

### BACKGROUND

In recent years, biotechnology firms and research institutions have improved hardware and software for sequencing nucleotides and determining variant calls for genomic samples. For instance, some existing nucleobase sequencing platforms determine individual nucleobases within sequences from genomic samples' cells by using conventional Sanger sequencing or by using sequencing-by-synthesis (SBS) methods. When using SBS, existing platforms can monitor millions to billions of nucleic acid polymers being synthesized in parallel to predict nucleobase calls from a larger base call dataset. For instance, a camera in many SBS platforms captures images of irradiated fluorescent tags incorporated into oligonucleotides for determining the nucleobase calls. After capturing such images, existing sequencing platforms send base call data (or image-based data) to a computing device to apply sequencing data analysis software that determines a nucleobase sequence for a genomic sample or other nucleic acid polymer. For instance, such software (i) maps and aligns nucleotide reads determined by the sequencing platform for a sample with (ii) a reference genome comprising at least a primary contiguous sequence. Based on differences between the aligned nucleotide reads and the reference genome, existing data analysis software can further utilize a variant caller to identify genotype and/or variants within a genomic sample, such as single nucleotide polymorphisms (SNPs), insertions or deletions (indels), or structural variants.

Despite these recent advances, existing nucleobase sequencing platforms and sequencing data analysis software (together and hereinafter, “existing sequencing systems”) often utilize reference genomes that misrepresent certain populations and foment inaccurate read alignment and mistaken variant calling. For example, some existing sequencing systems use a linear reference genome that purportedly represents a consensus or example of genes and other nucleotide sequences of an organism. But about 93% of the primary assembly for the most common linear human reference genome, GRCh38 from the Genome Reference Consortium, is based on libraries from only 11 individuals, with 70% of the linear human reference genome coming from 1 individual. Accordingly, many existing systems use a linear reference genome that does not represent certain populations or common variants.

To address this lack of genetic representation in linear reference genomes, some existing sequencing systems generate or use a graph reference genome. For example, some graph reference genomes include both a linear reference genome and graph augmentations, with multi-nucleobase codes representing SNPs and/or indels and alternate contiguous sequences representing alternative population haplotypes at given regions. In some cases, such graph reference genomes stack and index alternate contiguous sequences that can stretch relatively long nucleobase distances (e.g., hundreds to thousands of base pairs in length) and, consequently, include redundant reference nucleobases overlapping a same region.

While such graph reference genomes better account for some populations' genetics, the expanded representation of existing graph reference genomes is often bulky and consume considerable memory and computing resources to implement. Indeed, some existing graph reference genomes can include countless graph augmentations for SNPs, indels, and other variations from a significant number of alternate contiguous sequences representing various population haplotypes, including some population haplotypes of relatively low allele frequency (e.g., less than 1% in population frequency). These seemingly countless alternative paths can consume unnecessary memory and needlessly require exorbitant computing resources to navigate when conducting mapping and alignment of nucleotide reads for a genomic sample. Indeed, conventional graph reference genomes often increase the computer processing time for existing sequencing systems to determine whether to include or exclude matches to graph augmentations when making read alignment inferences. In some cases, an excessive number of candidate alignments can lead existing sequencing systems to limit the resources available for further alignment procedures, resulting in further inaccuracies due to incomplete consideration of potential alignments.

Additionally, some existing graph reference genomes include an exorbitant number of alternative paths for alleles that are similar to other genomic regions and paths in the graph reference genome. Consequently, existing sequencing systems can significantly increase the difficulty of predicting accurate degradations from alternative paths by undermining the distinctness and usefulness of a genomic region for mapping and alignment and by increasing confusion between multiple look-alike genomic regions. For example, some existing sequencing systems utilize seed extensions of exceeding length to effectively locate unique matches within the graph sequence genome for the read. Such excessive seed extensions are less sensitive and can be a detriment to alignment accuracy as potential matches are overlooked. Further still, when processing paired-end reads, existing sequencing systems often struggle to locate mate alignments that accurately represent both mates within a reasonable distance of one another, due to numerous overlapping alternate contiguous sequences within either or both of their respective genomic regions.

Indeed, these generic graph reference genomes—with an excessive number of alternative paths representing alternative contiguous sequences—frequently cause existing sequencing systems to misalign, incorrectly match, or miss call variants for a large number of samples as well as increase the chances of mismatched alignments with reads from a genomic sample. Due to having multiple look-alike population haplotypes that lift over a given genomic region of a primary contiguous sequence—and diminishing mapping quality (e.g., MAPQ 0) as such population haplotypes increase in number for the given genomic region—existing sequencing systems have often failed to scale up candidate population haplotypes in a graph reference genome without slowing computation time for mapping and aligning, reducing mapping quality, and reducing variant-calling accuracy.

These, along with additional problems and issues exist in existing sequencing systems.

### SUMMARY

This disclosure describes embodiments of methods, non-transitory computer-readable media, and systems that (i) determine primary alignment scores for read alignments with primary contiguous sequences and (ii) adjust the primary alignment scores based on comparisons between reads and allele-variant differences representing differences between the primary contiguous sequence and population haplotypes. In particular, the disclosed systems can identify candidate alignments between nucleotide reads from a genomic sample with a primary contiguous sequence at respective genomic regions of a reference genome. For each of the candidate alignments, the systems can identify allele-variant differences among the primary contiguous sequence and one or more population haplotypes corresponding to a respective genomic region of the reference genome. Based on the identified allele-variant differences, the systems generate adjustments to the respective primary alignment score. When a candidate alignment between nucleotide reads and a locally distinct population haplotype (as represented by one or more allele-variant differences) improves the alignment score, the systems can generate a replacement alignment score for such a candidate alignment with the locally distinct population haplotype. Based on the scoring of the candidate alignments, the disclosed systems can identify a candidate alignment exhibiting a superior primary alignment score or replacement alignment score and determine predicted read alignments for the respective nucleotide reads.

To facilitate such improved methods of mapping and alignment, the disclosed systems can utilize a haplotype data structure comprising a hierarchical partitioning of a reference genome's regions into reference bins representing respective genomic regions (e.g., spans of a set number of nucleobases) of the reference genome. For example, the disclosed haplotype data structure can include a base level having a set of base-level bins comprising respective base-level reference spans of a first length between respective genomic coordinates of the reference genome, where each base-level bin includes variant data for nucleotide variants of locally distinct population haplotypes within the corresponding genomic region. In addition to such base-level bins, the disclosed haplotype data structure can include successive levels of higher-level bins comprising respective higher level reference spans of a greater length than the base-level reference spans of the base-level bins, where each higher-level bin includes variant-data indices referencing combinations of the variant data from corresponding base-level bins from the set of base-level bins. By utilizing such a haplotype data structure to identify allele-variant differences among the primary contiguous sequence and locally distinct population haplotypes from the variant data stored and referenced within one or more bins corresponding to a candidate read alignment, the systems can generate alignment scores for a genomic sample's nucleotide reads to account for such allele-variant differences and select a predicted read alignment based on the corresponding scores for the candidate alignments.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which a read alignment adjustment system can operate in accordance with one or more embodiments of the present disclosure.

FIG. 2 illustrates an overview of an existing sequencing system conducting mapping and alignment of nucleotide reads from a genomic sample using a conventional graph reference genome with graph augmentations representing population haplotypes.

FIG. 3A illustrates the read alignment adjustment system determining candidate alignments between nucleotide reads with a primary contiguous sequence and generating primary alignment scores for the respective candidate alignments in accordance with one or more embodiments of the present disclosure.

FIG. 3B illustrates the read alignment adjustment system generating adjusted alignment scores based on allele-variant differences between a primary contiguous sequence and one or more population haplotypes in accordance with one or more embodiments of the present disclosure.

FIG. 4 illustrates the read alignment adjustment system determining alignment score adjustments for candidate alignments of paired-end nucleotide reads in accordance with one or more embodiments of the present disclosure.

FIG. 5A further illustrates the read alignment adjustment system determining candidate alignments for nucleotide reads and generating primary alignment scores for the candidate alignments in accordance with one or more embodiments of the present disclosure.

FIG. 5B illustrates the read alignment adjustment system generating a replacement alignment score for a given candidate alignment in accordance with one or more embodiments of the present disclosure.

FIG. 6 illustrates the read alignment adjustment system determining a replacement alignment score for a candidate alignment from a set of adjusted alignment scores in accordance with one or more embodiments of the present disclosure.

FIG. 7 illustrates a set of base-level bins of a haplotype data structure in accordance with one or more embodiments of the present disclosure.

FIG. 8 illustrates base-level bins and successive higher-level bins of the haplotype data structure in accordance with one or more embodiments of the present disclosure.

FIGS. 9A-9B illustrate experimental results of utilizing the haplotype data structure to encode variant data for a panel of population haplotypes in accordance with one or more embodiments of the present disclosure.

FIG. 10 illustrates the read alignment adjustment system utilizing the haplotype data structure to determine alignment score adjustments for a candidate alignment of a nucleotide read in accordance with one or more embodiments of the present disclosure.

FIG. 11 illustrates the read alignment adjustment system utilizing the haplotype data structure to determine and sum alignment score adjustments for a candidate alignment of a paired-end nucleotide read in accordance with one or more embodiments of the present disclosure.

FIG. 12 illustrates an example implementation of the read alignment adjustment system utilizing the haplotype data structure to determine alignment score adjustments for a candidate spliced alignment of a transcriptomic read in accordance with one or more embodiments of the present disclosure.

FIGS. 13A-13B illustrate comparative experimental results of determining variant calls from nucleotide reads that are (i) mapped and aligned with a reference genome using existing sequence systems and (ii) mapped and aligned to a reference genome using the read alignment adjustment system and the haplotype data structure in accordance with one or more embodiments of the present disclosure.

FIGS. 14A-14B illustrate example implementations of determining alignment scores for candidate alignments of nucleotide reads that are (i) mapped and aligned with a reference genome using existing sequencing systems and (ii) mapped and aligned to a reference genome using the read alignment adjustment system in accordance with one or more embodiments of the present disclosure.

FIG. 15 illustrates a flowchart of a series of acts for selecting a predicted read alignment for one or more nucleotide reads from a genomic sample in accordance with one or more embodiments of the present disclosure.

FIG. 16 illustrates a flowchart of a series of acts for utilizing a haplotype data structure to select a predicted read alignment for one or more nucleotide reads from a genomic sample in accordance with one or more embodiments of the present disclosure.

FIG. 17 illustrates a block diagram of an example computing device for implementing one or more embodiments of the present disclosure.

### DETAILED DESCRIPTION

This disclosure describes embodiments of a read alignment adjustment system that can utilize a haplotype data structure that encodes allele-variant differences to determine alignments of nucleotide reads from a genomic sample with a primary contiguous sequence of a reference genome or with a population haplotype represented by the allele-variant differences in the data structure. In particular, the read alignment adjustment system can utilize a haplotype data structure comprising graph augmentations that encode population variation in respective genomic regions to allow for scoring of candidate alignments without directly aligning reads to alternate contiguous sequences. For instance, the read alignment adjustment system can identify, for one or more nucleotide reads from a genomic sample, a set of candidate read alignments between the nucleotide reads with a primary contiguous sequence at a respective set of genomic regions of a reference genome and generate a primary alignment score for each candidate alignment. For each candidate read alignment, the read alignment adjustment system can determine alignment score adjustments to account for allele-variant differences in each locally distinct haplotype within the respective genomic region. Additionally, the read alignment adjustment system can adjust alignment scores for candidate alignments based on population frequencies of the respective locally distinct haplotypes.

As mentioned above, embodiments of the read alignment adjustment system can utilize a haplotype data structure encoding population variation within respective genomic regions of a reference genome to facilitate mapping and alignment according to the methods described herein. For example, the read alignment adjustment system can implement a haplotype data structure comprising a hierarchical partitioning of a reference genome into reference bins representing respective genomic regions (e.g., spans of nucleobases) of the reference genome and encoding allele-variant differences for locally distinct population haplotypes within the respective genomic regions.

To facilitate efficient alignment scoring of both primary contiguous sequences and locally distinct population haplotypes, the disclosed haplotype data structure can include a base level having a set of base-level bins comprising respective base-level reference spans of a first length between respective genomic coordinates of the reference genome, each base-level bin including variant data for nucleotide variants of locally distinct population haplotypes within the corresponding genomic region. In some cases, each base-level bin has a matrix including corresponding variant data representing allele-variant differences from locally distinct haplotypes and variant positions for the allele-variant differences.

In addition to such base-level bins, the disclosed haplotype data structure can include successive levels of higher-level bins comprising respective higher level reference spans of a greater length than the base-level reference spans of the base-level bins, each higher-level bin including variant-data indices referencing combinations of the variant data from corresponding base-level bins from the set of base-level bins. As described further below, in certain cases, each higher-level bin includes “offset” bins that cover different nucleobase spans than “non-offset” bins, such that every combination of two subsequent bins from the level below is represented by either a non-offset bin or an offset bin. To query a span of the reference genome, the read alignment adjustment system accesses a lowest-level bin containing an entire candidate alignment of a nucleotide read as well as the non-offset bins below the lowest-level bin.

Accordingly, in some embodiments, the read alignment adjustment system utilizes such a haplotype data structure to identify allele-variant differences among the primary contiguous sequence and locally distinct population haplotypes. By encoding such locally distinct population haplotypes in variant data stored and referenced within one or more bins corresponding to a candidate read alignment, the read alignment adjustment system performs one or more of the disclosed methods for mapping and alignment of nucleotide reads. In one or more embodiments, for example, the read alignment adjustment system can identify a bin of the haplotype data structure corresponding to a reference span that includes every nucleobase position in a candidate alignment of a nucleotide read, or multiple linked reads, from a genomic sample. Based on the variant data stored or indicated within the selected bin, the read alignment adjustment system can identify allele-variant differences for locally distinct population haplotypes within the corresponding reference span to determine alignment score adjustments for the candidate alignment to aid in selection of a predicted read alignment for the respective nucleotide read(s). When a candidate alignment between nucleotide reads and a locally distinct population haplotype (as represented by one or more allele-variant differences) improves the alignment score, for instance, the read alignment adjustment system generates a replacement alignment score for such a candidate alignment for the locally distinct population haplotype.

As suggested above, the read alignment adjustment system provides several technical advantages, benefits, and/or improvements over existing sequencing systems, including systems utilizing conventional graph reference genomes augmented with alternate contiguous sequences and other sequencing data analysis software. In some embodiments, for instance, the read alignment adjustment system can accurately predict read alignments while improving the computing speed and memory usage relative to existing sequencing systems. As noted above, existing sequencing systems use graph reference genomes with generic graph augmentations including numerous and redundant alternate contiguous sequences that consume memory with the repeated sequences from overlapping portions of alternate contiguous sequences and slow down computer processing by scoring alignments between reads and such overlapping portions of alternate contiguous sequences. In contrast to such existing systems, the disclosed read alignment adjustment system expedites determines alignment scores at least by: (i) adjusting alignment scores for candidate alignments between nucleotide reads and a primary contiguous sequence based on differences between population haplotypes and the primary contiguous sequence and (ii) providing a haplotype data structure representing allele-variant differences in genomic regions.

By determining alignment score adjustments for locally distinct population haplotypes based on allele-variant differences between a primary contiguous sequence and each locally distinct haplotype, for example, the disclosed methods can accurately determine predicted read alignments for nucleotide reads with improved computational speed and less memory relative to the graph genomes of existing sequencing systems. In particular, as mentioned above, existing sequencing systems often determine predicted read alignments by attempting to align and score nucleotide reads with a robust graph genome augmented by alternative contiguous sequences. Rather than determining alignment scores for alternate contiguous sequences that lift over the same given primary contiguous sequence—and often rescoring alignments between spans of the same sequence—the read alignment adjustment system expedites alignment scoring by first determining candidate alignments with a primary contiguous sequence then adjusting alignment scores for the candidate alignments based on differences between the primary contiguous sequence and alternate contiguous sequences of population haplotypes, which are encoded as allele-variant differences. The disclosed read alignment adjustment system, therefore, improves computing speed for mapping and aligning nucleotide reads of a genomic sample with a reference genome that represents alternate population haplotypes.

In addition to improved computing speed and reduced memory, by utilizing various embodiments of the haplotype data structure described herein, the read alignment adjustment system provides for accurate and comprehensive population-haplotype information in a scalable manner. As disclosed herein, for example, the haplotype data structure can readily be upscaled to include variation and frequency data for virtually any number of population haplotypes due to the minimal data storage required to encode population variations for locally distinct haplotypes in respective genomic regions without encoding nucleobases at base positions where there are no allele-variant differences between the respective haplotypes and the primary contiguous sequence. As depicted and described in this disclosure, for instance, the read alignment adjustment system can increase the number of population haplotypes represented in the disclosed haplotype data structure from 32 population haplotypes to 128 (or more) population haplotypes without compromising mapping accuracy or variant-calling accuracy.

Moreover, by initially mapping nucleotide reads to a primary contiguous sequence, as opposed to utilizing a graph reference genome additionally including numerous alternate contiguous sequences, the read alignment adjustment system enables improved methods for mapping and alignment. In some implementations, for example, haplotype nucleobases are encoded in the primary contiguous sequence (e.g., via multi-base coding) to increase seed mapping sensitivity in difficult-to-map regions. Also, when performing mapping and alignment of paired-end reads, rescue scans can be performed as needed by using the primary contiguous sequence to generate candidate alignments for respective mates of paired-end reads. Further, for such paired candidate mate alignments, the haplotype data structure can be queried with a reference span covering both mate alignments and the respective alignment score jointly adjusted for further improved accuracy in predicting read alignments.

As suggested by the foregoing discussion, this disclosure utilizes a variety of terms to describe features and benefits of the read alignment adjustment system and the improved haplotype data structure. Additional detail is hereafter provided regarding the meaning of these terms as used in this disclosure. As used in this disclosure, for instance, the term “genomic sample” (or simply “sample”) refers to a specimen, culture, or the like that is suspected of including a target nucleic acid. In some embodiments, the genomic sample comprises DNA, ribonucleic acid (RNA), peptide nucleic acid (PNA), locked nucleic acid (LNA), chimeric or hybrid forms of nucleic acids as targets. The genomic sample can likewise include any biological, clinical, surgical, agricultural-atmospheric, or aquatic-based specimen containing one or more nucleic acids. A genomic sample also includes any isolated or extracted nucleic acid sample from an organism, such a genomic DNA, fresh-frozen, or formalin-fixed paraffin-embedded nucleic acid specimen. In some cases, accordingly, a genomic sample includes a full genome that is isolated or extracted (e.g., in whole or in part by a kit) from an organism and that is prepared to undergo sequencing or an assay in a sequencing device. A genomic sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material, such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The genomic sample can include high molecular weight material, such as genomic DNA (gDNA). The genomic sample can include low molecular weight material such as nucleic acid molecules obtained from FFPE or archived DNA samples. In another implementation, low molecular weight material includes enzymatically or mechanically fragmented DNA. The genomic sample can include cell-free circulating DNA. In some implementations, the genomic sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the genomic sample can be an epidemiological, agricultural, forensic, or pathogenic sample. In some implementations, the genomic sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Also, as used herein, the term “nucleotide read” (or simply “read”) refers to an inferred or predicted sequence of one or more nucleotide bases (or nucleobase pairs) from all or part of a sample genomic sequence (e.g., a sample genomic sequence, complementary DNA). Such a sample nucleotide sequence may take the form of a sample genomic sequence from genomic DNA (gDNA), a transcriptomic sequence from complementary DNA (cDNA), a transcriptomic sequence from RNA, or other nucleotide sequence. In particular, a nucleotide read includes a determined or predicted sequence of nucleobase calls for a nucleotide fragment (or group of monoclonal nucleotide fragments) from a sequencing library corresponding to a genomic sample. For example, in some embodiments, a sequencing device determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

Relatedly, as used herein, the term “genomic read” refers to a nucleotide read representing an inferred sequence of nucleobases (or nucleobase pairs) derived from genomic DNA (gDNA) extracted from a sample. For example, a genomic read includes a read comprising gDNA that is (i) extracted from or derived from gDNA extracted from a sample and (ii) part of a sample library fragment corresponding to the sample.

Conversely, as used herein, the term “transcriptomic read” refers to a nucleotide read representing an inferred sequence of nucleobases (or nucleobase pairs) that either complement or represent RNA extracted from a sample. For example, a transcriptomic read includes a read comprising cDNA that is (i) synthesized from single-stranded messenger RNA (mRNA) or microRNA (miRNA) or derived from RNA extracted from a sample and (ii) part of a sample library fragment corresponding to the sample. As a further example, a transcriptomic read includes a read comprising RNA (e.g., mRNA, miRNA, transfer RNA (tRNA)) that is (i) extracted from or derived from RNA extracted from a sample and (ii) part of a sample library fragment corresponding to the sample.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a somatic or sex chromosome (e.g., chr1 or chrX) and a particular position or positions, such as numbered positions following the identifier for a chromosome (e.g., chr1:1234570 or chr1:1234570-1234870). In some cases, a genomic coordinate refers to a genomic coordinate on a sex chromosome (e.g., chrX or chrY). Consequently, the read alignment adjustment system can determine genotype probabilities for a genotype call (e.g., a variant call) for a genomic coordinate on a sex chromosome. Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

As used herein, a “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1:1234570-1234870). In various implementations, a genomic coordinate includes a position within a reference genome. In some cases, a genomic coordinate is specific to a particular reference genome. Relatedly, as used herein, the term “reference span” refers to a span of nucleobase positions within a linear reference genome. In other words, a reference span includes a span of nucleobases between two respective genomic coordinates of the linear reference genome.

As noted above, a genomic coordinate includes a position within a reference genome. Such a position may be within a particular reference genome. As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. As noted above, in some cases, a reference genome includes multi-base codes. As a further example, a reference genome may include a graph reference genome that includes both a linear reference genome and paths representing nucleic acid sequences from ancestral haplotypes, such as Illumina DRAGEN Graph Reference Genome hg19.

As used herein, the term “primary contiguous sequence” (or simply “primary contig”) refers to a contiguous sequence representing a reference haplotype of the reference genome. In some embodiments, a primary contiguous sequence digitally represents a reference haplotype of a reference genome but can include additional information from a primary assembly of the linear reference genome, such as indications of population variants in certain genomic regions to aid in identifying candidate alignments of nucleotide reads.

By contrast, the term “alternate contiguous sequence” (or simply “alt contig”) refers to a contiguous sequence representing an alternate population haplotype at particular genomic coordinates of a reference genome. For example, in some sequencing systems, a graph reference genome includes alternate contiguous sequences mapped to genomic coordinates of a primary assembly for a linear reference genome. In some cases, a hash table for a graph reference genome includes identifiers that associate alternate contiguous sequences representing population haplotypes at genomic coordinates relative to a linear reference genome. Critically, as explained and depicted in this disclosure, the disclosed haplotype data structure or corresponding reference genome does not directly include alternate contiguous sequences but rather encodes allele-variant differences between a primary contiguous sequence and locally distinct haplotypes within a given genomic region.

Relatedly, as used herein, the term “allele-variant difference” refers to differences between respective nucleobases of two or more given nucleotide sequences. In some cases, for example, allele-variant differences are differences between the primary contiguous sequence and at least one population haplotype (e.g., as represented by an alternative contiguous sequence). In some embodiments, for example, allele-variant differences within a given genomic region can include single nucleotide variants, multiple base differences, and/or insertions and deletions (indels) of population haplotypes relative to a primary contiguous sequence. Also, allele-variant differences can refer to differences between a first population haplotype and a second population haplotype.

As used herein, the term “haplotype data structure” refers to a data structure encoding variant data for population haplotypes of a sample organism. In particular, the haplotype data structure disclosed herein comprises a hierarchical partitioning of different genomic regions of a reference genome into a collection of bins covering respective spans of a linear reference genome (e.g., as represented by a primary contiguous sequence). Moreover, as used herein, the term “base-level bin” refers to a bin corresponding to a genomic region of a reference genome and encoding variant data for population haplotypes having allele-variant differences within the respective genomic region. For instance, in some cases, a base-level bin includes a region-specific data structure, such as a matrix, that encodes allele-variant differences from locally distinct population haplotypes for a given genomic region. Relatedly, as used herein, the term “base-level reference span” refers to a span of nucleobases of a genomic region to which a given base-level bin corresponds. As illustrated below, a base-level reference span represents or covers a number of nucleobases in a given genomic region of a reference genome, but does not need to represent each nucleobase in the given genomic region.

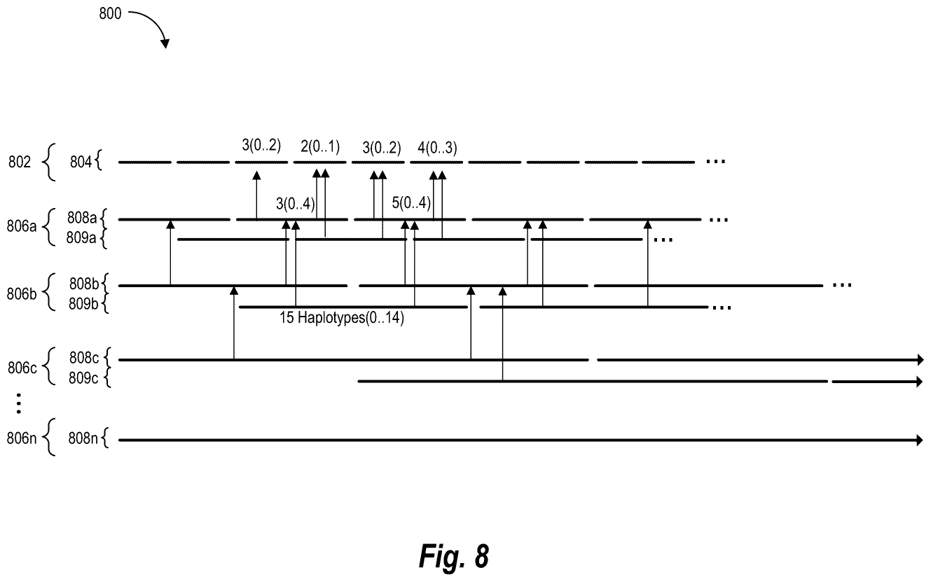

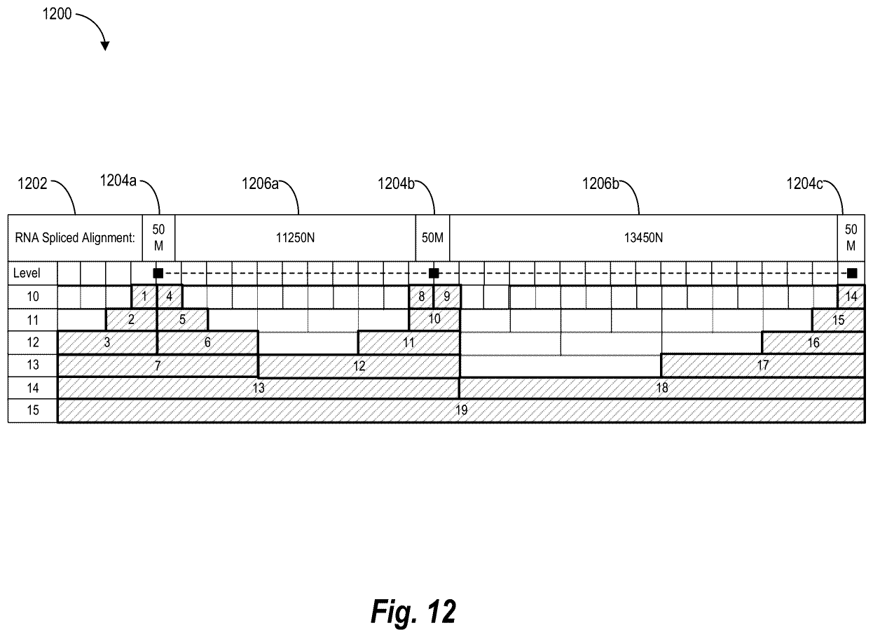

Further, as used herein, the term “higher-level bin” refers to a bin corresponding to an expanded genomic region of a greater length relative to respective base-level bins of a haplotype data structure. As illustrated below, a higher-level bin can include variant-data indices referencing combinations of variant data from corresponding base-level bins. Additionally or alternatively, in some cases, a higher-level bin can include variant-data indices referencing other variant-data indices within corresponding higher-level bins of a level below the respective higher-level bin, described below in relation to FIG. 12. Accordingly, a higher-level bin need not itself include variant data, but rather indices that identify variant data that encodes allele-variant differences. Relatedly, as used herein, the term “higher-level reference span” refers to a span of nucleobases of a genomic region to which a given higher-level bin corresponds. Also, as used herein, the term “variant-data indices” refers to encoded data within a given higher-level bin that references variant data within base-level bins corresponding to the given higher-level bin (e.g., as described in relation to FIGS. 8 and 12 below).

Also, as used herein, the term “locally distinct population haplotype” or “locally distinct haplotype” refers to a haplotype comprising a set of at least one allele-variant difference, where the set is unique relative to other haplotypes within a respective genomic region of a reference genome. Each bin of a haplotype data structure, according to the disclosed embodiments, for example, encodes one or more locally distinct haplotypes having a unique set of one or more allele-variant differences relative to other population haplotypes within each respective genomic region (e.g., as described in relation to FIG. 8 below). Also, in some embodiments, a given set of one or more allele-variant differences within a genomic region corresponding to a candidate read alignment can represent multiple haplotypes due to a complete overlap of variants within the genomic region. Accordingly, in certain cases, multiple haplotypes consisting of identical nucleobases within a given genomic region can be represented by a single locally distinct haplotype.

Moreover, as used herein, the term “alignment score” refers to a numeric score, metric, or other quantitative measurement evaluating an accuracy of an alignment between one or more nucleotide reads or a fragment of a nucleotide read and another nucleotide sequence from a reference genome. In particular, an alignment score includes a metric indicating a degree to which the nucleobases of one or more nucleotide reads (or a fragment thereof) match or are similar to a reference sequence or an alternate contiguous sequence from a reference genome. In certain implementations, an alignment score takes the form of a Smith-Waterman score or a variation or version of a Smith-Waterman score for local alignment, such as various settings or configurations used by DRAGEN by Illumina, Inc. for Smith-Waterman scoring.

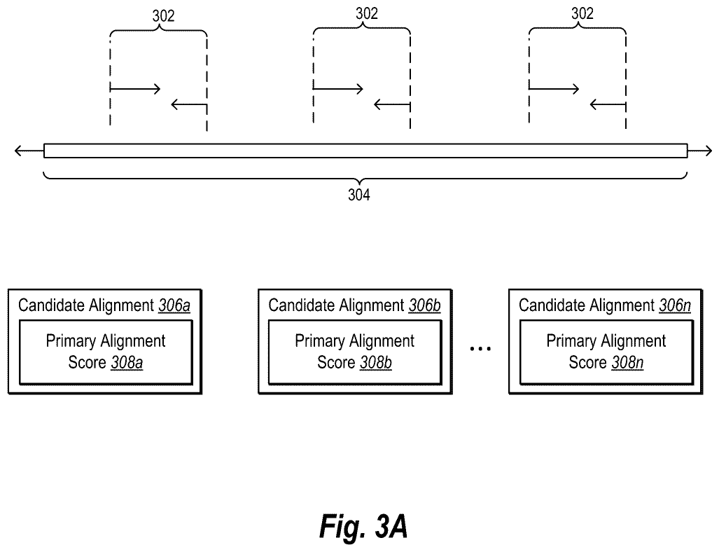

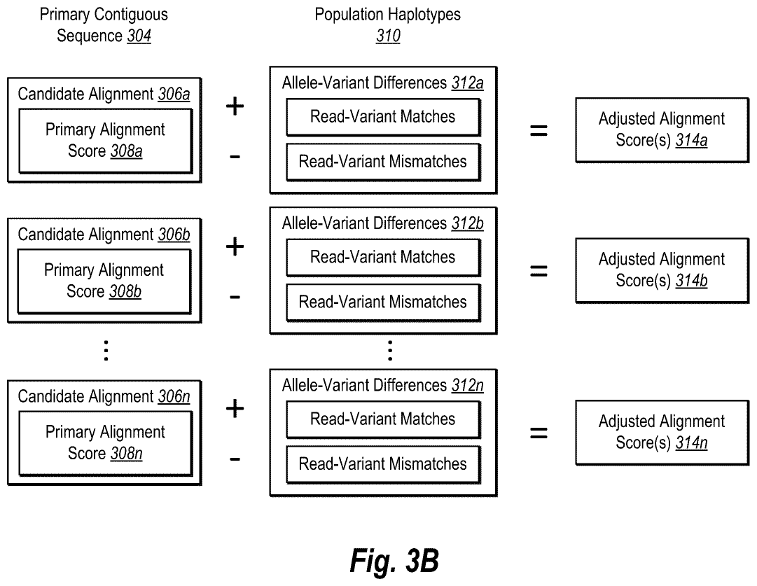

Relatedly, as used herein, the term “primary alignment score” refers to an alignment score generated for a candidate alignment between a nucleotide read and a primary contiguous sequence. Accordingly, in some cases, a primary alignment score does not account for population haplotypes within a genomic region corresponding to the candidate alignment. Also, as used herein, the term “adjusted alignment score” refers to an alignment score, for a given candidate alignment of a nucleotide read with a reference genome, that has been adjusted to account for allele-variant differences between a population haplotype and the primary contiguous sequence within a genomic region of the given candidate alignment (e.g., as described in relation to FIG. 3B below).

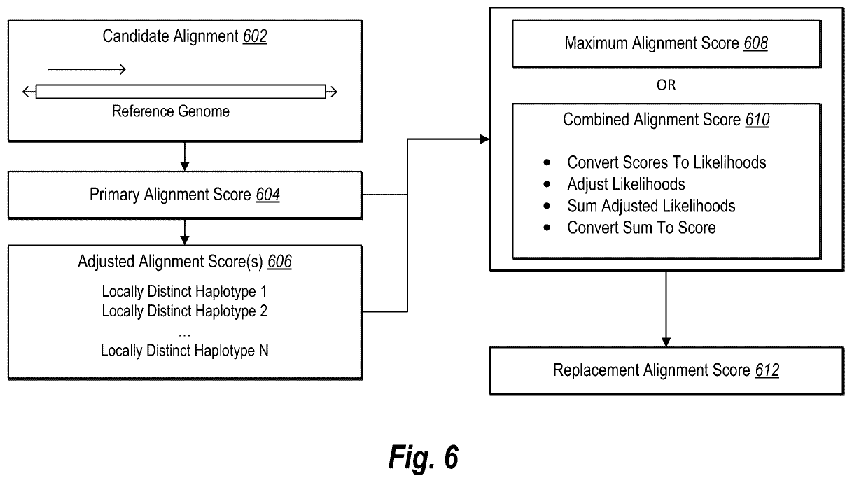

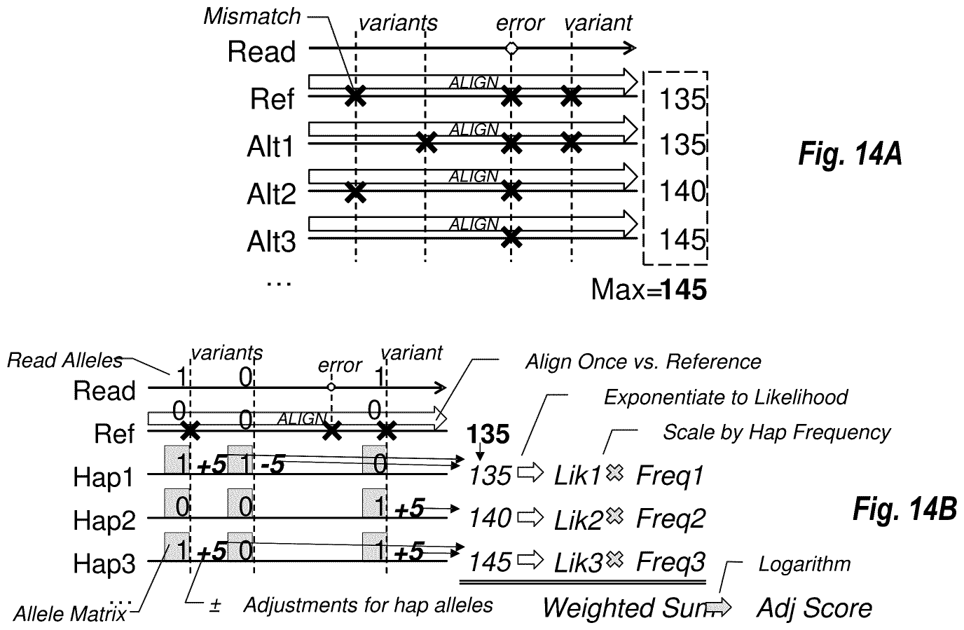

As further used herein, the term “replacement alignment score” refers to an alignment score, for a given candidate alignment of a nucleotide read with a reference genome, that has been generated to replace a primary alignment score for the given candidate alignment based on one or more adjusted alignments scores determined for the given candidate alignment in consideration of one or more population haplotypes within a genomic region of the given candidate alignment (e.g., as described in relation to FIG. 6 below). When a candidate alignment between nucleotide reads and a locally distinct population haplotype (as represented by one or more allele-variant differences) improves a primary alignment score, for instance, the read alignment adjustment system can generate a replacement alignment score for such a candidate alignment with the locally distinct population haplotype and rely on the replacement alignment score (instead of the primary alignment score) to determine whether the candidate alignment exhibits a highest relative alignment score and qualifies as a predicted read alignment for the nucleotide reads. As used herein, the terms “replacement alignment score” and “final adjusted alignment score” can be used interchangeably, such as in the description below for FIG. 14B.

Relatedly, as used herein, the term “mapping-quality score” refers to a metric or other measurement quantifying a quality or certainty of an alignment of nucleotide reads (or other nucleotide sequences or subsequences) with a reference genome. In some embodiments, for example, a mapping-quality score includes mapping quality (MAPQ) scores for nucleobase calls at genomic coordinates, where a MAPQ score represents −10 log 10 Pr{mapping position is wrong}, rounded to the nearest integer. In the alternative to a mean or median mapping quality, in some implementations, a mapping-quality score includes a full distribution of mapping qualities for all nucleotide reads aligning with a reference genome at a genomic coordinate.

As further used herein, the term “genotype call” refers to a determination or prediction of a particular genotype of a genomic sample or a sample nucleotide sequence at a genomic locus. In particular, a genotype call can include a prediction of a particular genotype of a genomic sample with respect to a reference genome or a reference sequence at a genomic coordinate or a genomic region. For instance, in some cases, a genotype call includes a determination or prediction that a genomic sample comprises both a nucleobase and a complementary nucleobase at a genomic coordinate that is either homozygous or heterozygous for a reference base or a variant (e.g., homozygous reference bases represented as 0|0 or heterozygous for a variant on a particular strand represented as 0|1). Accordingly, a genotype call can include a prediction of a variant or reference base for one or more alleles of a genomic sample and indicate zygosity with respect to a variant or reference base. A genotype call is often determined for a genomic coordinate or genomic region at which an SNP, insertion, deletion, or other variant has been identified for a population of organisms.

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate a determination or prediction of the type of nucleobase that has been incorporated within an oligonucleotide on a nucleotide-sample slide (e.g., read-based nucleobase calls). In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). As suggested above, a single nucleobase call can be an adenine (A) call, a cytosine (C) call, a guanine (G) call, a thymine (T) call, or an uracil (U) call.

As used herein, the term “variant” refers to a nucleobase or multiple nucleobases that do not align with, differ from, or vary from a corresponding nucleobase (or nucleobases) in a reference sequence or a reference genome. For example, a variant includes a SNP, an indel, or a structural variant that indicates nucleobases in a sample nucleotide sequence that differ from nucleobases in corresponding genomic coordinates of a reference sequence.

Along these lines, a “variant call” (or “variant nucleobase call”) refers to a nucleobase call comprising a mutation or a variant at a particular genomic coordinate or genomic region with respect to a reference. In particular, a variant call includes a determination or prediction that a genomic sample comprises a particular nucleobase (or sequence of nucleobases) at a genomic coordinate or region that differs from a reference nucleobase (or sequence of reference nucleobases) at the same genomic coordinate or region within a reference genome. Conversely, a “non-variant call” (or “non-variant nucleobase call” or “reference call”) refers to a nucleobase call comprising a non-variant or a reference nucleobase at a genomic coordinate or a genomic region with respect to a reference. In particular, a non-variant or reference call includes a determination or prediction that a genomic sample comprises a particular nucleobase (or sequence of nucleobases) at a genomic coordinate or region that matches a reference nucleobase (or sequence of reference nucleobases) at the same genomic coordinate or region within a reference genome.

In one or more embodiments, the read alignment adjustment system identifies and/or stores sequencing metrics within one or more sequencing data files. As used herein, the term “sequencing data file” refers to a digital file that includes genetic sequencing information concerning genotype calls or nucleotide reads generated by one or more genomic sequencing procedures. Such sequencing information may include, for example, nucleotide reads, alignment and mapping information, nucleotide reads at one or more genomic coordinates, and so forth.

Moreover, in one or more embodiments, one or more sequencing data files in which the read alignment adjustment system identifies or stores sequencing metrics include an alignment data file containing information from a read processing and mapping procedure. As used herein, the term “alignment data file” refers to a digital file that indicates mapping and alignment information for nucleotide reads of a sample nucleotide sequence. For example, an alignment data file can include a binary alignment map (BAM) file, a compressed reference-oriented alignment map (CRAM) file, or another file indicating nucleotide reads of a sample nucleotide sequence.

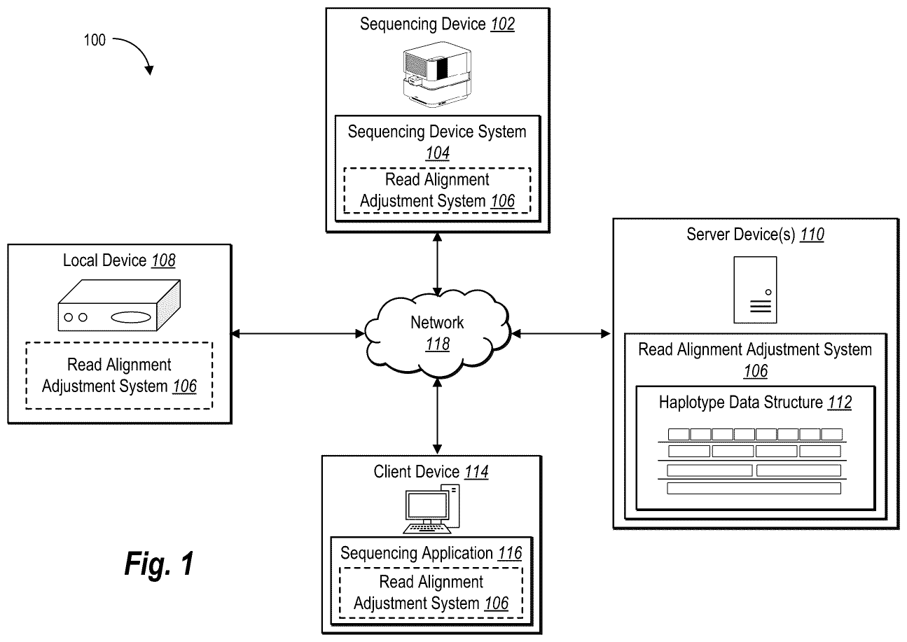

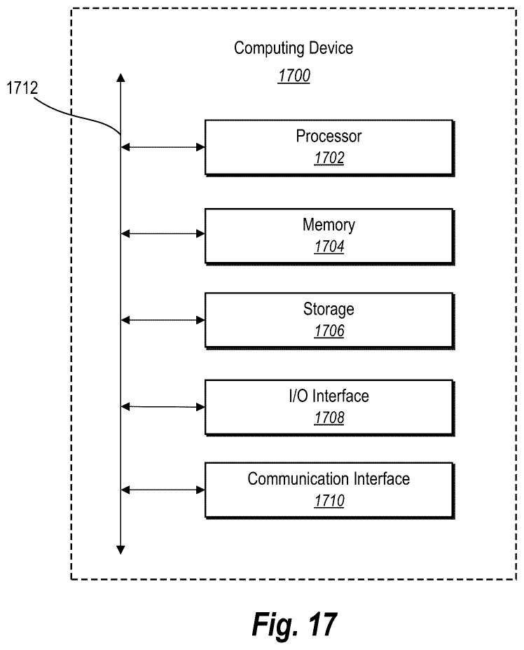

The following paragraphs describe the read alignment adjustment system with respect to illustrative figures that portray example embodiments and implementations. For example, FIG. 1 illustrates a schematic diagram of a computing system 100 in which a read alignment adjustment system 106 operates in accordance with one or more embodiments. As illustrated, the computing system 100 includes a sequencing device 102 connected to a local device 108 (e.g., a local server device), one or more server device(s) 110, and a client device 114. As shown in FIG. 1, the sequencing device 102, the local device 108, the server device(s) 110, and the client device 114 can communicate with each other via a network 118. The network 118 comprises any suitable network over which computing devices can communicate. Example networks are discussed in additional detail below with respect to FIG. 17. While FIG. 1 shows an embodiment of the read alignment adjustment system 106, this disclosure describes alternative embodiments and configurations below.

As indicated by FIG. 1, the sequencing device 102 comprises a computing device and a sequencing device system 104 for sequencing a genomic sample or other nucleic-acid polymer. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments extracted from samples and further copies and determines the nucleobase sequence of such extracted nucleotide fragments.

In one or more embodiments, the sequencing device 102 utilizes sequencing-by-synthesis (SBS) techniques to sequence nucleotide fragments into nucleotide reads and determine nucleobase calls for the nucleotide reads. In addition or in the alternative to communicating across the network 118, in some embodiments, the sequencing device 102 bypasses the network 118 and communicates directly with the local device 108 or the client device 114. By executing the sequencing device system 104, the sequencing device 102 can further store the nucleobase calls as part of base-call data that is formatted as a binary base call (BCL) file and send the BCL file to the local device 108 and/or the server device(s) 110.

As further indicated by FIG. 1, the local device 108 is located at or near a same physical location of the sequencing device 102. Indeed, in some embodiments, the local device 108 and the sequencing device 102 are integrated into a same computing device. The local device 108 may run the read alignment adjustment system 106 to generate, receive, analyze, store, and transmit digital data, such as by receiving base-call data or determining variant calls based on analyzing such base-call data. As shown in FIG. 1, the sequencing device 102 may send (and the local device 108 may receive) base-call data generated during a sequencing run of the sequencing device 102. By executing software in the form of the read alignment adjustment system 106, the local device 108 may align nucleotide reads with a reference genome utilizing a haplotype data structure 112 and determine genetic variants based on the aligned nucleotide reads. The local device 108 may also communicate with the client device 114. In particular, the local device 108 can send data to the client device 114, including a binary alignment map (BAM) file, a variant call format (VCF) file, or other information indicating nucleobase calls, sequencing metrics, error data, or other metrics.

As further indicated by FIG. 1, the server device(s) 110 are located remotely from the local device 108 and the sequencing device 102. Similar to the local device 108, in some embodiments, the server device(s) 110 include a version of (or are otherwise able to access or implement) the read alignment adjustment system 106. Accordingly, the server device(s) 110 may generate, receive, analyze, store, and transmit digital data, such as by receiving base-call data or determining variant calls based on analyzing such base-call data. As indicated above, the sequencing device 102 may send (and the server device(s) 110 may receive) base-call data from the sequencing device 102. The server device(s) 110 may also communicate with the client device 114. In particular, the server device(s) 110 can send data to the client device 114, including BAM files, VCF files, or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As indicated above, as part of the server device(s) 110 or the local device 108, the read alignment adjustment system 106 can generate, encode, and/or implement the haplotype data structure 112 to determine alignments of nucleotide reads from a genomic sample with a reference genome. For instance, the read alignment adjustment system 106 can identify candidate alignments of one or more nucleotide reads with a primary contiguous sequence, generate primary alignment scores for the candidate alignments, and adjust the alignment scores based on population variant data indicated in the haplotype data structure 112, as described in greater detail below in relation to the subsequent figures.

As further illustrated and indicated in FIG. 1, by executing a sequencing application 116, the client device 114 can generate, store, receive, and send digital data. In particular, the client device 114 can receive sequencing data from the local device 108 or receive call files (e.g., BCL) and sequencing metrics from the sequencing device 102. Furthermore, the client device 114 may communicate with the local device 108 or the server device(s) 110 to receive a VCF comprising genotype or variant calls and/or other metrics, such as a base-call-quality metrics or pass-filter metrics. The client device 114 can accordingly present or display information pertaining to variant calls or other genotype calls within a graphical user interface of the sequencing application 116 to a user associated with the client device 114. For example, the client device 114 can present genotype calls, variant calls, and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 17.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the read alignment adjustment system 106 and present, for display at the client device 114, base-call data or data from a VCF. Furthermore, the sequencing application 116 can instruct the client device 114 to display summaries for multiple sequencing runs.

As further illustrated in FIG. 1, a version of the read alignment adjustment system 106 may be located and/or implemented (e.g., entirely or in part) on the client device 114 or the sequencing device 102. In yet other embodiments, the read alignment adjustment system 106 is implemented by one or more other components of the computing system 100, such as the local device 108. In particular, the read alignment adjustment system 106 can be implemented in a variety of different ways across the sequencing device 102, the local device 108, the server device(s) 110, and the client device 114. For example, the read alignment adjustment system 106 can be downloaded from the server device(s) 110 to the read alignment adjustment system 106 and/or the local device 108 where all or part of the functionality of the read alignment adjustment system 106 is performed at each respective device within the computing system 100.

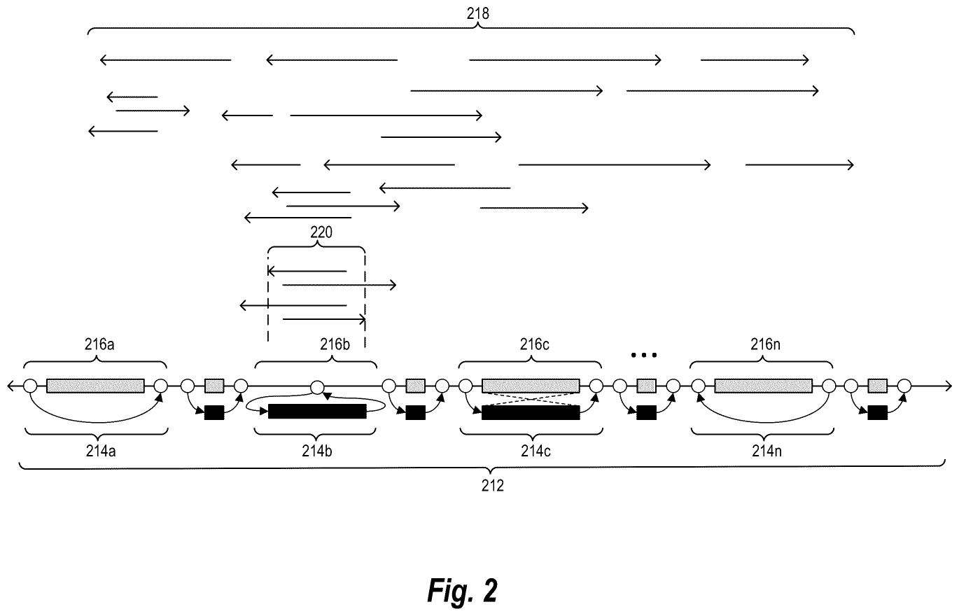

As previously mentioned, in some embodiments, the read alignment adjustment system 106 implements and/or utilizes an improved haplotype data structure encoding allele-variant differences between a primary contiguous sequence and population haplotypes across a linear reference genome. In contrast, as also mentioned, some existing sequencing systems utilize graph reference genomes including both a linear reference genome and graph augmentations representing alternate contiguous sequences having SNPs and/or indels. To illustrate, FIG. 2 depicts an example of an existing sequencing system aligning nucleotide reads of a genomic sample with a graph reference genome 212 for determining nucleobase calls for the genomic sample based on the aligned nucleotide reads.

As shown in FIG. 2, for example, the depicted sequencing system identifies or receives nucleotide reads 218 for a genomic sample and aligns the nucleotide reads 218 with different sequences of the graph reference genome 212. As can sometimes be the case with graph reference genomes, the graph reference genome 212 includes a linear reference genome comprising reference sequences 216a, 216b, 216c through 216n augmented by various alternate contiguous sequences 214a, 214b, 214c through 214n representing various population haplotypes in relation to the linear reference genome. As indicated by the ellipsis (or dots) in FIG. 2, the graph reference genome 212 can include more reference sequences and/or more alternate contiguous sequences than those depicted in FIG. 2. While FIG. 2 depicts the alternate contiguous sequences 214a-214n as not overlapping with each other, in some cases, a graph reference genome utilized by existing sequencing systems includes numerous overlapping alternate contiguous sequences with lift over at any given genomic region of the linear reference genome. Accordingly, existing sequencing systems, such as the depicted system in FIG. 2, must often consider numerous alternate contiguous sequences in addition to a linear reference sequence when mapping and aligning nucleotide reads to a graph reference genome.

As illustrated, for example, the depicted sequencing system predicts alignment of a subset of nucleotide reads 220 from the nucleotide reads 218 with the alternate contiguous sequence 214b of the graph reference genome 212. As FIG. 2 suggests, at least some of the subset of nucleotide reads 220 overlap with the alternate contiguous sequence 214b. While not shown in FIG. 2, individual nucleotide reads (or related groupings of nucleotide reads) often overlap with multiple sequences included in a graph reference genome, such as the graph reference genome 212 depicted in FIG. 2. For example, in addition to aligning with the alternate contiguous sequence 214b, the subset of nucleotide reads 220 would likely overlap (at least partially) with one or more other alternate contiguous sequences (not shown) of the graph reference genome 212 and with the reference sequences 216b of the graph reference genome 212, and/or with one or more multi-base codes not depicted in FIG. 2.

As noted above, in some embodiments, the read alignment adjustment system 106 determines candidate alignments between nucleotide reads from a genomic sample and a primary contiguous sequence and evaluates the candidate alignments based on variations between the primary contiguous sequence and respective population haplotypes. FIGS. 3A-3B, for example, illustrate the read alignment adjustment system 106 determining candidate alignments 306a, 306b through 306n for nucleotide reads 302 and, based on population haplotypes 310, generating adjusted alignment scores 314a, 314b through 314n from respective primary alignment scores 308a, 308b through 308n corresponding to the candidate alignments 306a, 306b through 306n. In describing FIGS. 3A-3B, the following paragraphs give an overview of the read alignment adjustment system 106 (i) determining primary alignment scores for read alignments with primary contiguous sequences and (ii) adjusting the primary alignment scores based on comparisons between reads and allele-variant differences representing differences between the primary contiguous sequence and population haplotypes. As indicated by the ellipsis (or dots) in FIGS. 3A and 3B, the read alignment adjustment system 106 can identify, determine, generate, or utilize more candidate alignments, primary alignment scores, allele-variant differences, and/or adjusted alignment score(s) than those depicted in FIGS. 3A and 3B. After describing FIGS. 3A-3B, this disclosure provides further detail and embodiments of the read alignment adjustment system 106 in subsequent paragraphs and figures.

In one or more embodiments, for example, the read alignment adjustment system 106 identifies or receives nucleotide reads for a genomic sample. In some cases, for instance, the read alignment adjustment system 106 receives base-call data (e.g., BCL file(s) or FASTQ file(s)) from a sequencing device, which has sequenced oligonucleotides extracted from the genomic sample and determined individual nucleobase calls for the nucleotide reads in the base-call data. Depending on the type of sequencing performed, in some embodiments, the read alignment adjustment system 106 identifies or receives either single-end reads or paired-end reads and either relatively short nucleotide reads (e.g., <300 base pairs or <10,000 base pairs) or relatively long nucleotide reads (e.g., >300 base pairs or >10,000 base pairs) for mapping and alignment with a reference genome.

As shown in FIG. 3A, the read alignment adjustment system 106 aligns a subset of nucleotide reads 302 from a genomic sample with a primary contiguous sequence 304 at different genomic regions of a reference genome to determine the candidate alignments 306a-306n. To illustrate but a few candidate regions for alignment, FIG. 3A depicts the subset of nucleotide reads 302 at three different genomic regions corresponding to candidate alignments 306a, 306b, and 306n. In one or more embodiments, for example, the primary contiguous sequence 304 includes a linear reference sequence comprising an accepted representation of a reference genome (e.g., a human genome) corresponding to the genomic sample. In some implementations, the primary contiguous sequence 304 is selectively augmented to include data representing population variation in certain genomic regions of the reference genome. For example, the primary contiguous sequence 304 can include multi-base coded nucleotide positions representing population variation in regions determined to be difficult to map (e.g., genomic regions comprising population variations at relatively high frequencies within a reference population).

As illustrated, the read alignment adjustment system 106 generates the primary alignment scores 308a-308n for the respective candidate alignments 306a-306n based on a comparison of nucleobases within the subset of nucleotide reads 302 with nucleobases indicated by the primary contiguous sequence 304 at respective genomic regions of the candidate alignments 306a-306n. In some embodiments, the read alignment adjustment system 106 identifies candidate alignments 306a-306n having respective alignment scores with respect to the primary contiguous sequence 304 that exceed a threshold alignment score for selection as a candidate alignment. In some embodiments, for example, the read alignment adjustment system 106 utilizes a Smith-Waterman score, a modified version of a Smith-Waterman score, or a similar scoring model or standard to generate the primary alignment scores 308a-308n with respect to the primary contiguous sequence 304.

Furthermore, as mentioned above, the read alignment adjustment system 106 adjusts the primary alignment scores 308a-308n for each of the respective candidate alignments 306a-306n based on population variation at the respective genomic regions of the reference genome. As shown in FIG. 3B, for example, the read alignment adjustment system 106 generates one or more adjusted alignment score(s) 314a-314n for each of the respective candidate alignments 306a-306n based on comparing nucleobases within the subset of nucleotide reads 302 with variant nucleobases of the population haplotypes 310 at the genomic regions of the respective candidate alignments 306a-306n.

In particular, as illustrated in FIG. 3B, the read alignment adjustment system 106 identifies allele-variant differences 312a, 312b through 312n between the primary contiguous sequence 304 and the population haplotypes 310 with respect to the respective candidate alignments 306a, 306b through 306n. Based on the allele-variant differences 312a-312n, the read alignment adjustment system 106 determines adjustments to the corresponding primary alignment scores 308a-308n and generates an adjusted alignment score for each population haplotype (or each locally distinct population haplotype) comprising variations at the respective genomic regions of the reference genome. For example, the allele-variant differences 312a-312n between the population haplotypes 310 and the primary contiguous sequence 304 can include any type of variant, such as, but not limited to, single nucleotide polymorphisms (SNPs), insertions or deletions (indels), or other structural variants.

As further shown in FIG. 3B, the read alignment adjustment system 106 identifies, for each respective genomic region of the candidate alignments 306a-306n, the allele-variant differences 312a-312n between the primary contiguous sequence 304 and the population haplotypes 310. Based on the allele-variant differences 312a-312n, the read alignment adjustment system 106 determines, for each population haplotype of the population haplotypes 310 that includes one or more variations from the primary contiguous sequence 304, an adjusted alignment score.

For example, for the candidate alignment 306a, the read alignment adjustment system 106 identifies allele-variant differences 312a corresponding to one or more population haplotypes within the respective genomic region of the reference genome. From the allele-variant differences 312a for each of the one or more population haplotypes comprising variants within the respective genomic region, the read alignment adjustment system 106 determines one or more adjusted alignment scores of the adjusted alignment score(s) 314a corresponding to the one or more population haplotypes. In particular, in some embodiments, the read alignment adjustment system 106 increases the primary alignment score 308a for each match between a nucleobase of the nucleotide reads 302 and a variant nucleobase of a given haplotype of the population haplotypes 310, as represented by the allele-variant difference 312a. Further, the read alignment adjustment system 106 decreases the primary alignment score 308a for each mismatch between a nucleobase of the nucleotide reads 302 and a variant nucleobase of a given haplotype of the population haplotypes 310, as represented by the allele-variant difference 312a. Accordingly, as shown in FIG. 3B, the read alignment adjustment system 106 generates an adjusted alignment score of the adjusted alignment score(s) 314a corresponding to each identified haplotype of the population haplotypes 310 in the respective genomic region of the candidate alignment 306a. Moreover, the read alignment adjustment system 106 performs similar steps to determine one or more adjusted alignment scores 314b-314n from the primary alignment scores 308b-308n for the remaining candidate alignments 306b-306n.

In some embodiments, in addition to alignment score adjustments based on read-variant matches and/or mismatches between the nucleotide reads 302 and the respective population haplotypes 310, the read alignment adjustment system 106 further adjusts the primary alignment scores 308a-308b based on a population frequency (e.g., a population allele frequency) of the respective population haplotypes 310. For example, the read alignment adjustment system 106 can increase a respective adjusted alignment score for a population haplotype having a relatively high frequency within a reference population or decrease a respective adjusted alignment score for a population haplotype having a relatively low frequency within a reference population.

Accordingly, as shown in FIG. 3B, the read alignment adjustment system 106 can generate multiple adjusted alignment scores 314a-314n for each of the respective candidate alignments 306a-306n. Based on the primary alignment scores 308a-308n and the respective adjusted alignment scores 314a-314n, the read alignment adjustment system 106 can select a predicted alignment of the nucleotide reads 302 with a respective genomic region of the reference genome represented by the primary contiguous sequence 304. For example, as described in additional detail below (e.g., in relation to FIG. 6), the read alignment adjustment system 106 can generate a replacement alignment score for one or more of the candidate alignments 306a, 306b, or 306n based on the respective primary alignment scores 308a, 308b, or 308n, respectively, and the adjusted alignment scores 314a, 314b, or 314n, respectively, and, based on the replacement alignment scores, select a predicted alignment of the nucleotide reads 302 (e.g., by selecting the candidate alignment with the highest replacement alignment score).

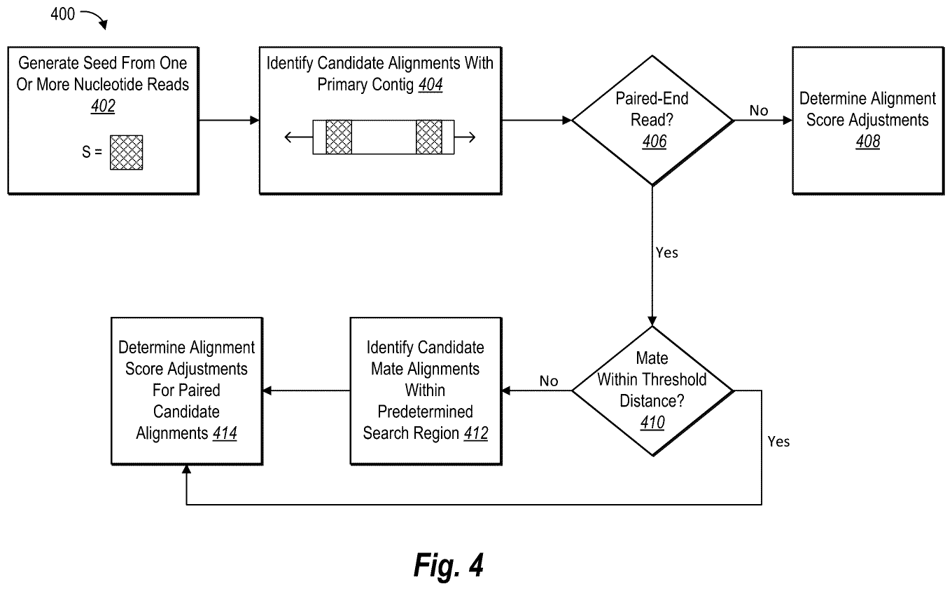

As mentioned previously, in one or more embodiments, the read alignment adjustment system 106 determines alignment scores for one or more nucleotide reads, including single-end nucleotide reads, paired-end reads, or otherwise grouped nucleotide reads from a genomic sample. For example, FIG. 4 illustrates an overview of a series of acts 400 for determining alignment score adjustments for unpaired reads and/or for paired-end reads. In various embodiments, the read alignment adjustment system 106 performs one or more actions from the series of acts 400 shown in FIG. 4.

As shown, the series of acts 400 includes an act 402 of generating a seed from one or more nucleotide reads. For instance, the read alignment adjustment system 106 identifies one or more nucleotide reads corresponding to a genomic region of a genomic sample. For example, the read alignment adjustment system 106 may identify nucleotide reads corresponding to a sample genomic sequence of a genomic sample. More specifically, a sample genomic sequence comprises a contiguous DNA or RNA fragment that is isolated or extracted from a sample organism and used as a template to sequence or produce complementary copies in the form of nucleotide reads by either single-end or paired-end methods. Accordingly, the sample genomic sequence is sometimes referred to as a template or template sequence. In the single-end method, a single-end nucleotide read is sequenced from one end (or a primer) of the sample genomic sequence. Because the single-end nucleotide read is sequenced from one end of the sample genomic sequence, the single-end nucleotide read represents the complementary sequence of the sample genomic sequence.

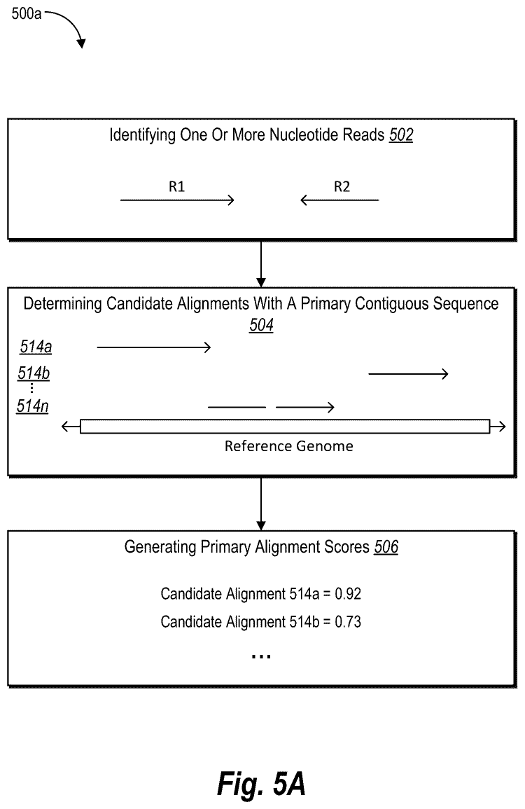

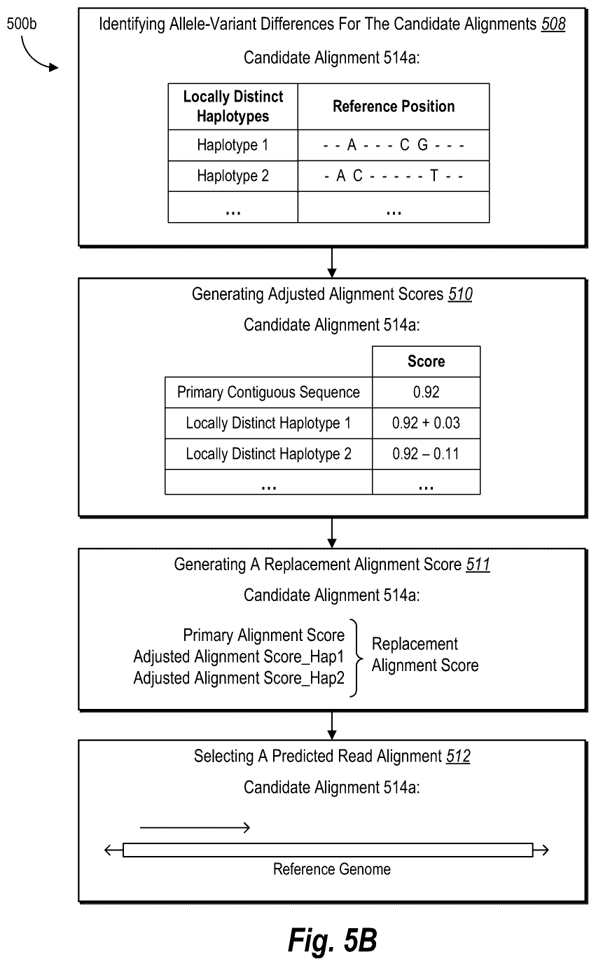

By contrast, in the paired-end method, a first nucleotide read (e.g., R1) is sequenced from one end (or a first primer) of the sample genomic sequence toward the middle and a second nucleotide read (e.g., R2) is sequenced from the other end (or second primer). This disclosure provides further examples of first and second nucleotide reads in FIG. 5A, where reads R1 and R2 are oriented toward each other. As discussed herein, two paired-end nucleotide reads (e.g., R1 and R2) are generally referred to as mates. In some cases, there is a gap between two mates of paired-end nucleotide reads, whereas in other cases an overlap between mates of paired-end nucleotide reads can occur. As illustrated in the series of acts 400, the read alignment adjustment system 106 generates a k-mer (i.e., a nucleotide sequence of length k) seed based on the nucleobases indicated by the one or more nucleotide reads. To illustrate, the read alignment adjustment system 106 generates the seed S shown as a cross-hatched pattern in FIG. 4.

FIG. 4 also shows an act 404 of identifying candidate alignments with a primary contiguous sequence of a reference genome. For example, in various embodiments, the read alignment adjustment system 106 utilizes the seed to identify subsequences of the primary contiguous sequence which overlap, in whole or in part, with the one or more nucleotide reads utilized to generate the seed. As shown, the read alignment adjustment system 106 utilizes the seed to determine candidate locations along the primary contiguous sequences that match the nucleobases of the one or more nucleotide reads. In some implementations, the read alignment adjustment system 106 requires an exact match with the seed. In other implementations, the read alignment adjustment system 106 selects candidate alignments that match by a threshold number or fraction of nucleobases.

As further illustrated, the series of acts 400 includes an act of determining whether the one or more nucleotide reads comprise a paired-end read or, in other words, whether a nucleotide read corresponding to a candidate alignment is a mate of a paired-end read. If the nucleotide read is a single-end read (or otherwise unpaired), the read alignment adjustment system 106 performs an act 408 of determining alignment score adjustments for the single-end read, according to one or more embodiments described herein (see, e.g., FIG. 3B and the corresponding text).

In implementations comprising a paired-end read (e.g., as determined or identified in the act 406), by contrast, the series of acts 400 includes an act 410 of determining whether a candidate alignment of a first mate of the paired-end read is within a threshold distance (i.e., separated by less than a threshold number of nucleobases of the primary contiguous sequence) of a second mate of the paired end read. Accordingly, in some embodiments, the read alignment adjustment system 106 identifies one or more paired candidate alignments for the mates of a paired-end read.

As illustrated in FIG. 4, for instance, the series of acts 400 also includes an act 412 of identifying candidate mate alignments within a predetermined search region (e.g., a search region defined by a threshold number of nucleobases). In particular, when the read alignment adjustment system 106 determines, at act 410, that a second mate of a paired-end read is not within a threshold distance of a corresponding first mate, the read alignment adjustment system 106 can search for candidate alignments of the second mate within a search region defined by the threshold distance. Indeed, in some implementations, the read alignment adjustment system 106 can thus identify candidate mate alignments that would otherwise be overlooked (e.g., due to incomplete overlap with the primary contiguous sequence) by accounting for the pairing of paired-end reads.

For the paired candidate alignments that are already within the threshold distance, in various embodiments, the read alignment adjustment system 106 proceeds to an act 414 of determining alignments score adjustments for the candidate alignments. Otherwise, upon identifying candidate mate alignments within the predetermined search region (at act 412), the read alignment adjustment system 106 can perform the act 414 to determine alignment score adjustments for the paired candidate mate alignments. Thus, in one or more embodiments, the read alignment adjustment system 106 scores the paired candidate mate alignments together to generate adjusted alignments scores corresponding to the paired-end read.

As mentioned previously, in one or more embodiments, the read alignment adjustment system 106 generates adjusted alignments scores for candidate alignments of nucleotide reads with respective genomic regions of a reference genome based on one or more locally distinct haplotypes at the respective genomic regions. In accordance with one or more embodiments, FIGS. 5A-5B illustrate a series of acts 500a-500b for determining adjusted alignment scores for candidate alignments based on locally distinct haplotypes and generating, based on the adjusted alignment scores, a replacement alignment score for each candidate alignment.

As shown in FIG. 5A, for instance, the series of acts 500a includes an act 502 of identifying one or more nucleotide reads. As discussed above (e.g., in relation to FIG. 4), the one or more nucleotide reads can include single-end nucleotide reads, paired-end nucleotide reads, or other subsets of nucleotide reads from a genomic sample. As shown in FIG. 5A, for example, the read alignment adjustment system 106 can identify mates R1 and R2 of a paired-end nucleotide read for mapping and alignment with a primary contiguous sequence of a reference genome. As mentioned, however, the read alignment adjustment system 106 can perform the disclosed methods for mapping and alignment of single-end reads, paired-end reads, or otherwise grouped reads, such as a pileup of nucleotide reads from a genomic sample (e.g., as shown in FIG. 3A).

As also shown in FIG. 5A, the series of acts 500a includes an act 504 of determining candidate alignments 514a-514n between the one or more nucleotide reads and a primary contiguous sequence within respective genomic regions of a reference genome. As illustrated, for instance, the read alignment adjustment system 106 determines the candidate alignments 514a-514n of the nucleotide read R1 with a primary contiguous sequence, wherein the candidate alignments 514a-514n comprise various degrees of overlap with respective nucleobases of the primary contiguous sequence. For example, the candidate alignment 514b as shown comprises a shorter read length relative to the candidate alignment 514b due at least in part to the candidate alignment 514b overlapping with a shorter span of nucleobases of the primary contiguous sequence. Also, the candidate alignment 514n as shown comprises a split in the corresponding read, thus illustrating a partial alignment comprising a non-continuous overlap with the primary contiguous sequence. Indeed, the read alignment adjustment system 106 can determine candidate alignments of nucleotide reads having various degrees and configurations of overlap with the primary contiguous sequence. As indicated by the ellipsis (or dots) in FIGS. 5A and 5B, the read alignment adjustment system 106 can identify, determine, generate, or utilize more candidate alignments, primary alignment scores, allele-variant differences, locally distinct haplotypes, adjusted alignment score(s), replacement alignment score(s), and/or predicted read alignments than those depicted in FIGS. 5A and 5B.

As further shown in FIG. 5A, the series of acts 500a includes an act 506 of generating primary alignment scores for the candidate alignments of the one or more nucleotide reads with the primary contiguous sequence. For example, the read alignment adjustment system 106 generates an alignment score for each of the candidate alignments 514a-514n based on the amount of overlap between the one or more nucleotide reads and the primary contiguous sequence at the respective genomic regions of the reference genome. In some embodiments, the primary alignment scores comprise a Smith-Waterman score, an adjusted Smith-Waterman score, or an analogous scoring standard. As illustrated, for example, the read alignment adjustment system 106 determines a primary alignment score of 0.92 for the candidate alignment 514a and a primary alignment score of 0.73 for the candidate alignment 514b.

Having generated primary alignment scores for the candidate alignments 514a-514n, as further shown in FIG. 5B, the read alignment adjustment system 106 can perform the series of acts 500b to generate a replacement alignment score for one or more candidate alignments. As shown in FIG. 5B, for example, the series of acts 500b includes an act 508 of identifying allele-variant differences for the candidate alignments of the one or more nucleotide reads. To illustrate, in the implementation shown, the read alignment adjustment system 106 identifies at least two locally distinct haplotypes in a genomic region corresponding to the candidate alignment 514a, indicated as “Haplotype 1” and “Haplotype 2,” respectively. As shown, the read alignment adjustment system 106 identifies allele-variant differences for each respective locally distinct haplotype without express identification of reference nucleobases within each respective haplotype. In other words, in one or more embodiments, the read alignment adjustment system 106 identifies differences between locally distinct population haplotypes and the primary contiguous sequence without identifying matching nucleobases between the alternate contiguous sequences and the primary contiguous sequence. As indicated above, the read alignment adjustment system 106 thereby avoids comparing and determining alignment scores for nucleotide reads directly with alternate contiguous sequences.

In various embodiments, a particular population haplotype is “locally distinct” within a given genomic region of the reference genome (e.g., within a genomic region corresponding to a candidate alignment) if the population haplotype includes a unique set of variants (e.g., SNPs or indels) relative to other population haplotypes within the given genomic region of the reference genome. In implementations wherein two or more population haplotypes include an identical set of variants within the given genomic region, for example, the read alignment adjustment system 106 identifies just one locally distinct haplotype rather than two or more identical population haplotypes within the given genomic region. Also, in implementations wherein two given haplotypes have one or more identical variants within a given genomic region but also have at least one differing variant within the given genomic region, the read alignment adjustment system 106 identifies the two given haplotypes as separate locally distinct haplotypes.

As also shown in FIG. 5B, the series of acts 500b includes an act 510 of generating adjusted alignment scores corresponding to each identified population haplotype of the locally distinct haplotypes for each respective candidate alignment of the one or more nucleotide reads. Having previously generated a primary alignment score relative to the primary contiguous sequence for the candidate alignment 514a, for instance, the read alignment adjustment system 106 adjusts the primary alignment score based on the allele-variant differences identified for each locally distinct haplotype. By performing such adjustments to primary alignment scores, the read alignment adjustment system 106 generates adjusted alignment scores corresponding to the respective locally distinct haplotypes.

In particular, as also described above (e.g., in relation to FIG. 3B), the read alignment adjustment system 106 increases the primary alignment score when a nucleobase of a given haplotype matches that of the respective nucleotide read (e.g., as shown with respect to Locally Distinct Haplotype 1) and decreases the primary alignment score when a nucleobase of a given haplotype mismatches the respective nucleotide read (e.g., as shown with respect to Locally Distinct Haplotype 2). In various embodiments, the read alignment adjustment system 106 considers additional information when adjusting the primary alignment scores for each locally distinct haplotype, such as, but not limited to, population allele frequencies from each considered haplotype.

In one or more embodiments, for example, the read alignment adjustment system 106 further adjusts the primary alignment score for a given candidate alignment based on prior probabilities of haplotype variants (e.g., to reduce false positives in variant calls from reads aligned to rare haplotypes). Accordingly, in some embodiments, the read alignment adjustment system 106 identifies a population frequency (e.g., prior probability) for each allele-variant difference of each locally distinct population haplotype and determines alignment score adjustments that account for the relative rarity of each allele-variant difference. When the read alignment adjustment system 106 identifies an allele-variant difference with a relatively low prior probability, for example, the read alignment adjustment system 106 can reduce the adjusted alignment score corresponding to the respective haplotype, relative to the primary alignment score. Moreover, when the read alignment adjustment system 106 identifies an allele-variant difference with a relatively high prior probability, the read alignment adjustment system 106 can increase the adjusted alignment score accordingly.

Alternatively, in some embodiments, the read alignment adjustment system 106 initially determines adjusted alignment scores for locally distinct haplotypes within a genomic region corresponding to a given candidate read, then further adjusts each adjusted alignment score to account for the prior probability of each respective population haplotype. In one or more embodiments, for example, the read alignment adjustment system 106 converts the initial adjusted alignment scores to likelihoods (e.g., as discussed in relation to FIG. 6 below), then increases or decreases the resultant likelihoods based on the prior probabilities (e.g., the population frequencies) of the respective population haplotypes (e.g., increasing a given likelihood based on a relatively high population frequency or decreasing a given likelihood based on a relatively low population frequency).

Further, in some embodiments, the read alignment adjustment system 106 utilizes the primary alignment score and adjusted alignment scores for a given candidate alignment to determine a replacement alignment score for the given candidate alignment. For example, the series of acts 500b includes an act 511 of generating a replacement alignment score for one or more candidate alignments. To illustrate, as shown in FIG. 5B, the read alignment adjustment system 106 generates a replacement alignment score for the candidate alignment 514a based on the corresponding primary alignment score, the adjusted alignment score for Locally Distinct Haplotype 1, the adjusted alignment score for Locally Distinct Haplotype 2, and any additional adjusted alignment scores not depicted in FIG. 5B. Additional detail regarding replacement alignment scores is provided below in relation to FIG. 6.

As further shown in FIG. 5B, the series of acts 500b includes an act 512 of selecting a predicted read alignment for the one or more nucleotide reads. In some embodiments, the read alignment adjustment system 106 can select a predicted read alignment from the candidate read alignments 514a-514n based on replacement alignment scores generated for each candidate read alignments according to the series of acts 500a and 500b or, alternatively, based on a primary alignment score when the primary alignment score outperforms or exceeds a corresponding adjusted alignment score. As illustrated, for example, the read alignment adjustment system 106 selects the first candidate read alignment 514a from the candidate read alignments 514a-514n as having a highest corresponding replacement alignment score and, in some embodiments, outputs the candidate alignment 514a as the predicted read alignment for the one or more nucleotide reads. In some implementations, the read alignment adjustment system 106 can select multiple candidate read alignments for output (e.g., to a BAM file), such as but not limited to inconclusive cases of identical or nearly identical replacement alignment scores among multiple candidate alignments.

As mentioned, in some embodiments, the read alignment adjustment system 106 generates a replacement alignment score for a candidate alignment of one or more nucleotide reads based on a respective primary alignment score and one or more adjusted alignment scores generated according to the disclosed methods. In accordance with one or more embodiments, FIG. 6 illustrates the read alignment adjustment system 106 generating a replacement alignment score 612 for a candidate alignment 602 based on a corresponding primary alignment score 604 and adjusted alignment scores 606.

As shown in FIG. 6, the read alignment adjustment system 106 determines the primary alignment score 604 for the candidate alignment 602 of one or more nucleotide reads from a genomic sample with a primary contiguous sequence at a respective genomic region of a reference genome. Based on one or more locally distinct haplotypes within the respective genomic region, the read alignment adjustment system 106 determines the adjusted alignment scores 606 (e.g., as described above in relation to FIG. 3B). In the illustrated example shown in FIG. 6, for instance, the read alignment adjustment system 106 determines the adjusted alignment scores for each respective population haplotype of locally distinct haplotypes 1 through N. As indicated by the ellipsis (or dots) in FIG. 6, the read alignment adjustment system 106 can determine adjusted alignment scores for more locally distinct haplotypes than those depicted in FIG. 6.

As further illustrated in FIG. 6, the read alignment adjustment system 106 determines the replacement alignment score 612 for the candidate alignment 602 based on the primary alignment score 604 and the adjusted alignment scores 606. In various embodiments, the read alignment adjustment system 106 can utilize a variety of methods for determining the replacement alignment score 612 for the candidate alignment 602. For instance, in some implementations, the read alignment adjustment system 106 selects a maximum alignment score 608 from among the adjusted alignment scores 606 and the primary alignment score 604. In such embodiments, the maximum alignment score 608 constitutes the replacement alignment score 612.

By contrast, in some implementations, the read alignment adjustment system 106 determines a combined alignment score 610 based on the primary alignment score 604 and the adjusted alignment scores 606. In one or more embodiments, the read alignment adjustment system 106 converts each of the primary alignment score 604 and the adjusted alignment scores 606 into likelihoods (e.g., a quantified probability that the one or more nucleotide reads correspond to the respective primary or locally distinct population haplotype). In such embodiments, the combined alignment score 610 constitutes the replacement alignment score 612. For example, in some embodiments, the read alignment adjustment system 106 converts each alignment score to a likelihood according to the following mathematical relationship:

$$score = {log}_{∝} ( likelihood ) + C \\ likelihood = {∝}^{score - C}$$

wherein C represents a normalizing constant and ∝ represents a base selected according to length of the one or more nucleotide reads. Accordingly, as shown in FIG. 6, the read alignment adjustment system 106 converts the respective alignment scores to likelihoods and adjusts and/or combines the resulting likelihoods to determine an overall likelihood for the candidate alignment 602. In some cases, accordingly, the resulting replacement alignment score 612 represents a likelihood that the respective nucleotide read(s) correspond to the respective genomic region of the reference genome. By converting the overall/summed likelihood to an alignment score, the read alignment adjustment system 106 can generate the replacement alignment score 612 for the candidate alignment 602.

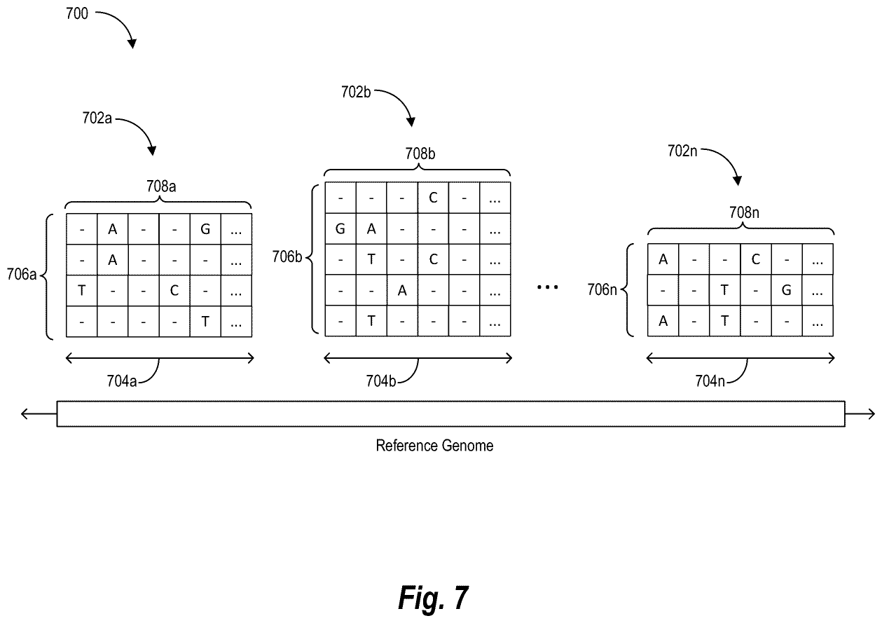

As mentioned previously, the read alignment adjustment system 106 can utilize an enhanced haplotype data structure that encodes allele-variant differences to implement the foregoing mapping and alignment methods. In accordance with one or more embodiments, FIGS. 7-8 illustrate a haplotype data structure comprising a hierarchical partitioning of a reference genome for efficient and accurate encoding of population haplotype data for a reference genome. In particular, FIG. 7 illustrates a base level of a haplotype data structure 700 according to one or more embodiments, and FIG. 8 illustrates a base level 802 and multiple successive levels 806a-806n of a haplotype data structure 800 according to one or more embodiments.

As shown in FIG. 7, the haplotype data structure 700 includes at least a base level comprising a set of base-level bins 702a, 702b through 702n that partition genomic regions of a reference genome into a respective set of base-level reference spans 704a, 704b through 704n. In one or more embodiments, each base-level reference span of the set of base-level reference spans 704a-704n comprises a genomic region of a first length between respective genomic coordinates of the reference genome, thus partitioning genomic regions of the reference genome into multiple bins spanning an equal portion/length of the reference genome. In various implementations, the length of the base-level reference spans can approximate, for example, the average or maximum length of nucleotide reads provided to the read alignment adjustment system 106 for mapping and alignment. Alternatively, the base-level reference spans can otherwise be selected to span a predetermined number of nucleobases from genomic coordinates or regions of a linear reference sequence, such as, but not limited to, 100 base pairs or 1000 base pairs per base-level bin.

As further illustrated in FIG. 7, the set of base-level bins 702a-702n of the haplotype data structure 700 comprise encoded variant data for nucleotide variants from respective sets of locally distinct haplotype(s) 706a-706n. As mentioned previously, each locally distinct haplotype within a given base-level bin comprises a unique set of one or more allele-variant differences relative to other population haplotypes also having variations within the genomic region of the respective base-level reference span of the given base-level bin. As shown in FIG. 7, for example, each row of the set of locally distinct haplotypes 706a comprises a unique set of allele-variant differences (denoted as single letters representing particular nucleotides) relative to other rows, such that no two rows are identical—although there can be limited overlap between allele-variant differences, as indicated by the top two rows of the base-level bin 702a. Accordingly, in one or more embodiments, population haplotypes having identical nucleotide variants within a given base-level bin are encoded as one locally distinct haplotype within the given base-level bin.

In various embodiments, each base-level bin of the haplotype data structure 700 can include differing quantities of locally distinct haplotypes. As shown in FIG. 7, for example, the base-level bin 702a includes four locally distinct haplotypes in the set of locally distinct haplotypes 706a (as indicated by the four rows of the portrayed matrix), the base-level bin 702b includes five locally distinct haplotypes in the set of locally distinct haplotypes 706b, and the base-level bin 702n includes three locally distinct haplotype in the set of locally distinct haplotypes 706n. Indeed, each base-level bin of the haplotype data structure 700 can include any number of locally distinct haplotypes, including as many as every population haplotype in a data set or no population haplotypes (e.g., in cases where there are no haplotypes having allele-variant differences in a genomic region corresponding to a given bin).

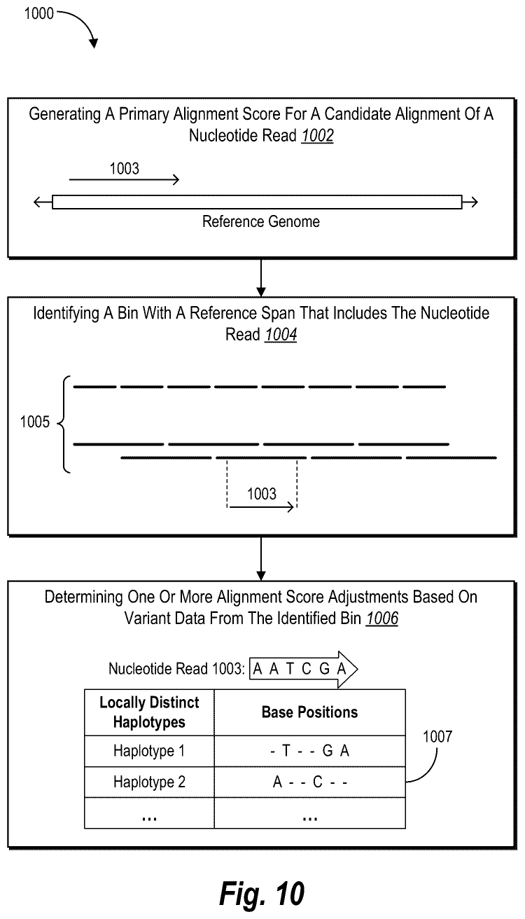

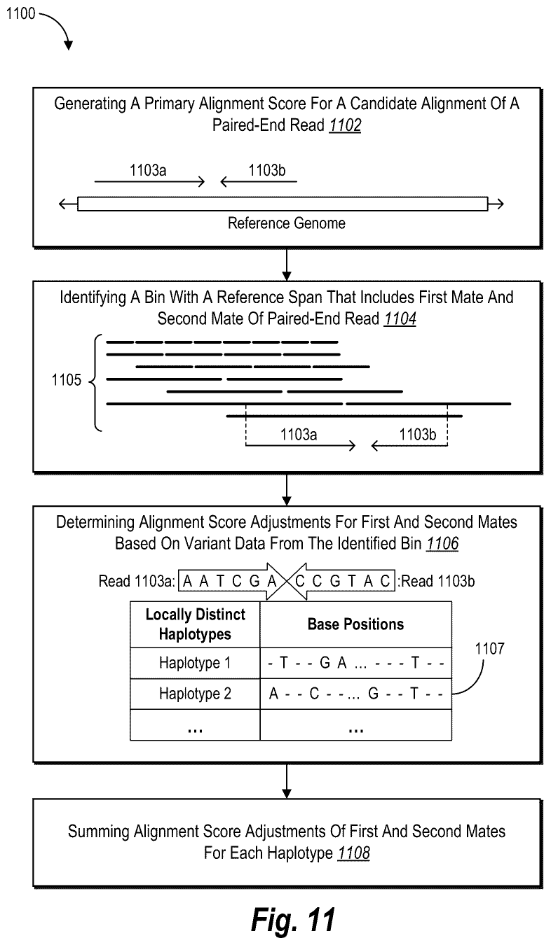

As further shown in FIG. 7, the set of base-level bins 702a, 702b through 702n include allele-variant differences 708a, 708b through 708n for each locally distinct haplotype of the respective sets of locally distinct haplotype(s) 706a, 706b through 706n. For example, variant data encoded within the base-level bin 702a includes one or more locally distinct population haplotypes of the set of locally distinct haplotypes 706a for which allele-variant differences 708a are included for each respective locally distinct haplotype. In some embodiments, for example, each base-level bin (e.g., of the set of base-level bins 702a-702n) comprises a matrix including corresponding variant data representing allele-variant differences from locally distinct haplotypes (e.g., of the respective sets of locally distinct haplotypes 706a-706n) and variant positions for the allele-variant differences (e.g., as illustrated in FIGS. 10-11). In various embodiments, the variant data within each base-level bin includes data indications (e.g., the allele-variant differences 708a-708n) of single-nucleotide polymorphisms (SNPs) and/or insertions or deletions (indels) at respective genomic coordinates of the reference genome (e.g., of the primary contiguous sequence). As indicated by the ellipsis (or dots) in FIG. 7, the read alignment adjustment system 106 can identify, determine, generate, or utilize more base-level bins, locally distinct population haplotypes, base-level reference spans, and/or allele-variant differences than those depicted in FIG. 7.

Moreover, in some embodiments, the base-level bins (e.g., the set of base-level bins 702a-702n) include the variant data for nucleotide variants without including reference nucleobases of the primary contiguous sequence. As shown in FIG. 7, for example, each base-level bin of the set of base-level bins 702a-702n comprises a matrix with rows representing the sets of locally distinct haplotypes 706a-706n within the corresponding set of base-level reference spans 704a-704n and columns representing the allele-variant differences 708a-708n of each respective locally distinct haplotype. As shown, allele-variant differences 708a-708n are indicated as letters representing nucleotides that differ from the primary contiguous sequence. Alternatively, in various embodiments, allele-variant differences can be indicated by numbers (e.g., with “0” indicating a nucleobase matching the primary contiguous sequence and subsequent values representing variations from the primary contiguous sequence), or similar means for representing differences between each population haplotype and the primary contiguous sequence.

As mentioned, in some embodiments, the read alignment adjustment system 106 utilizes a haplotype data structure with a hierarchical partitioning of genomic regions of a reference genome into multiple levels of bins corresponding to spans of nucleobases within the reference genome. For example, FIG. 8 illustrates a haplotype data structure 800 having a base level 802 comprising a set of base-level bins 804 and multiple successive levels 806a, 806b, 806c through 806n of higher-level bins spanning successively larger spans of nucleobases of a reference genome. Specifically, the haplotype data structure 800 comprises the base level 802 of the set of base-level bins 804 jointly spanning a primary contiguous sequence of the reference genome and the multiple successive levels 806a-806c of higher-level bins 808a-808c and offset higher-level bins 809a-809c also spanning the primary contiguous sequence of the reference genome. As indicated by FIG. 8, the successive level 806n comprises a higher-level bin 808n and a corresponding offset higher-level bin, but FIG. 8 does not depict the corresponding offset higher-level bin due to constraints on figure space. As further indicated by the ellipsis (or dots) in FIG. 8, the read alignment adjustment system 106 can identify, determine, generate, or utilize more base-level bins, successive levels, higher-level bins, and/or offset higher-level bins than those depicted in FIG. 8.

As illustrated, the base level 802 of the haplotype data structure 800 includes the set of base-level bins 804 corresponding to a respective set of base-level reference spans of the primary contiguous sequence for the reference genome. Each reference span of the set of base-level reference spans corresponds to a genomic region of a first length between respective genomic coordinates of the reference genome. In one or more embodiments, for example, each reference span of the set of base-level reference spans includes 1000 base pairs (1 kbp) of the primary contiguous sequence for the reference genome. Alternatively, the first length of the base-level reference spans can be less than or greater than 1 kbp, such as, but not limited to, 250 bp, 500 bp, 1500 bp, 5 kbp, 10 kpb, and so forth. Accordingly, in various embodiments, the set of base-level bins 804 collectively span either the entire primary contiguous sequence or a genomic region of interest, such as but not limited to an entire chromosome.

As further indicated by FIG. 8, the set of base-level bins 804 of the base level 802 comprise variant data for nucleotide variants from respective sets of locally distinct population haplotypes (e.g., as described above in relation to FIG. 7). As mentioned, each locally distinct population haplotype comprises a unique set of one or more allele-variant differences relative to other population haplotypes within a respective base-level reference span of a given base-level bin of the set of base-level bins 804. As shown in FIG. 8, for example, the set of base-level bins 804 comprise respective sets of locally distinct population haplotypes with varying numbers of locally distinct haplotypes, as indicated by the numbers associated with each base-level reference span of the set of base-level bins 804. As illustrated, for instance, a first base-level bin includes three locally distinct haplotypes (indicated by “3(0 . . . 2)”), a second base-level bin includes two locally distinct haplotypes (indicated by “2(0 . . . 1)”), a third base-level bin includes three locally distinct haplotypes (indicated by “3(0 . . . 2)”), and a fourth base-level bin includes four locally distinct haplotypes (indicated by “4(0 . . . 3)”). As mentioned previously, each locally distinct haplotype within a given base-level bin can represent one or more population haplotypes, as population haplotypes having identical nucleotide variants within a given base-level bin are encoded as one locally distinct population haplotype within the given base-level bin.

As also shown in FIG. 8, the haplotype data structure 800 comprises the multiple successive levels 806a-806n of higher-level bins 808a-808n. A first successive level 806a, for instance, comprises a first set of higher-level bins 808a corresponding to a first set of higher-level reference spans of the primary contiguous sequence for the reference genome. Each reference span of the first set of higher-level reference spans corresponds to an expanded genomic region of a second length between respective genomic coordinates of the reference genome, wherein the expanded genomic regions are expanded relative to the genomic regions represented by the set of base-level reference spans such that the second length (of the respective first set of higher-level reference spans) is longer than the first length (of the set of base-level reference spans). More specifically, as illustrated in FIG. 8, each higher-level bin of the first set of higher-level bins 808a of the first successive level 806a corresponds to a consecutive pair of the base-level bins in the set of base-level bins 804 from the base level 802 of the haplotype data structure 800.

Furthermore, as indicated by FIG. 8, the multiple successive levels 806a-806c of the haplotype data structure 800 comprise respective sets of offset higher-level bins 809a-809c and the successive level 806n of the haplotype data structure 800 comprises the higher-level bin 808n and a corresponding offset higher-level bin. For instance, the first successive level 806a includes a set of offset higher-level bins 809a corresponding to a first set of offset higher-level reference spans of the primary contiguous sequence for the reference genome. Each reference span of the first set of offset higher-level reference spans corresponds to an offset expanded genomic region of the second length (i.e., the same length as the reference spans of the first set of successive reference spans) between respective genomic coordinates of the reference genome. In like manner as the first set of higher-level bins 808a, the first set of offset higher-level bins 809a correspond to respective consecutive pairs of the base-level bins in the set of base-level bins 804 from the base level 802 of the haplotype data structure 800. Further, as illustrated, the respective reference spans of the first set of offset higher-level bins 809a are offset relative to the reference spans of the first set of higher-level bins 808a, such that each consecutive pair of the base-level bins in the set of base-level bins 804 is represented by either a higher-level bin or an offset higher-level bin from the first successive level 806a.

Moreover, each additional successive level 806b-806n of the haplotype data structure 800 comprises additional higher-level bins 808b-808n corresponding to respective additional higher-level reference spans corresponding to further expanded genomic regions between genomic coordinates of the primary contiguous sequence for the reference genome. In particular, as shown in FIG. 8, each higher-level bin (or offset higher-level bin) of a given successive level of the haplotype data structure 800 spans a combined genomic region of a pair of consecutive bins of a prior level of the haplotype data structure 800 (e.g., as indicated by the arrows linking various bins in FIG. 8). For example, the first illustrated bin of the set of higher-level bins 808c spans the same genomic region represented by the first two illustrated bins of the set of higher-level bins 808b. Likewise, the first illustrated bin of the set of higher-level bins 808b spans the same genomic region represented by the first two illustrated bins of the set of higher-level bins 808a. Indeed, each successive level comprises higher-level bins corresponding to a pair of consecutive bins from the previous level of the haplotype data structure 800.

Moreover, in some embodiments, the respective higher-level bins of each successive level of the haplotype data structure 800 comprise variant-data indices referencing combinations of the variant data from corresponding base-level bins of the base level 802. In particular, each higher-level bin and offset higher-level bin of the multiple sets of higher-level bins 808a-808c and offset higher-level bins 809a-809c, respectively—and each of the higher-level bin 808n and a corresponding offset higher-level bin—comprise variant-data indices referencing combinations of variant data from corresponding base-level bins of the set of base-level bins 804. Furthermore, the variant-data indices include indications of locally distinct haplotypes within each respective higher-level bin or offset higher-level bin. As illustrated in FIG. 8, for example, one of the offset higher-level bins 809b of the successive level 806b indicates fifteen locally distinct haplotypes (indicated by “15 Haplotypes (0 . . . 14)”). As also illustrated, two bins of the higher-level bins 808a from the previous successive level (e.g., the first successive level 806a) indicate three locally distinct haplotypes (indicated by “3(0 . . . 2)”) and five locally distinct haplotypes (indicated by “5(0 . . . 4)”), respectively. Additionally, in some embodiments, each bin of the haplotype data structure encodes population frequency data for each respective locally distinct haplotype therein (e.g., frequency of occurrence within a sample population of each locally distinct haplotype indicated within a given bin).

In one or more embodiments, the higher-level bins of each successive level comprise variant-data indices indicating locally distinct haplotypes and linking the higher-level bins to variant data within the corresponding base-level bins without including the variant data from the respective base-level bins, thus avoiding redundant encoding of variant data within the haplotype data structure. Referring to the successive level 806b, for example, the aforementioned bin of offset higher-level bins 809b indicating fifteen locally distinct haplotypes can include variant-data indices referencing how the locally distinct haplotypes of the corresponding higher-level bins (within the higher-level bins 808a) from the previous successive level (e.g., the first successive level 806a) combine to form the fifteen locally distinct haplotypes of the aforementioned bin. Further, each of the corresponding higher-level bins 808a can include variant data-indices referencing the locally distinct haplotypes (and the variant data thereof) indicated within the corresponding base-level bins (of the set of base-level bins 804) from the base level 802. Thus, by referencing variant-data indices within previous successive levels of the haplotype data structure 800, the variant-data indices of higher-level bins within the successive levels 806b-806n can also reference the variant data encoded within the set of base-level bins 804.

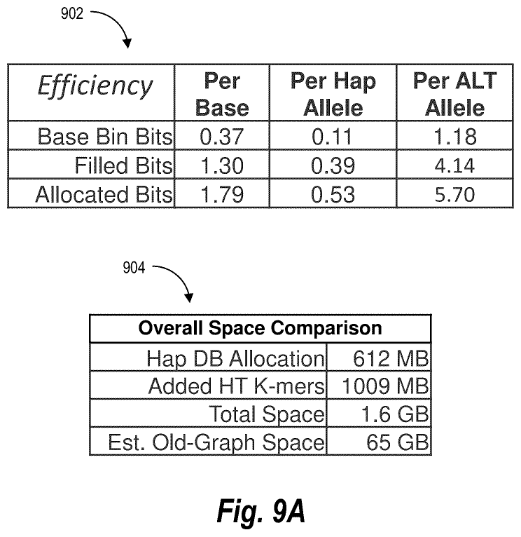

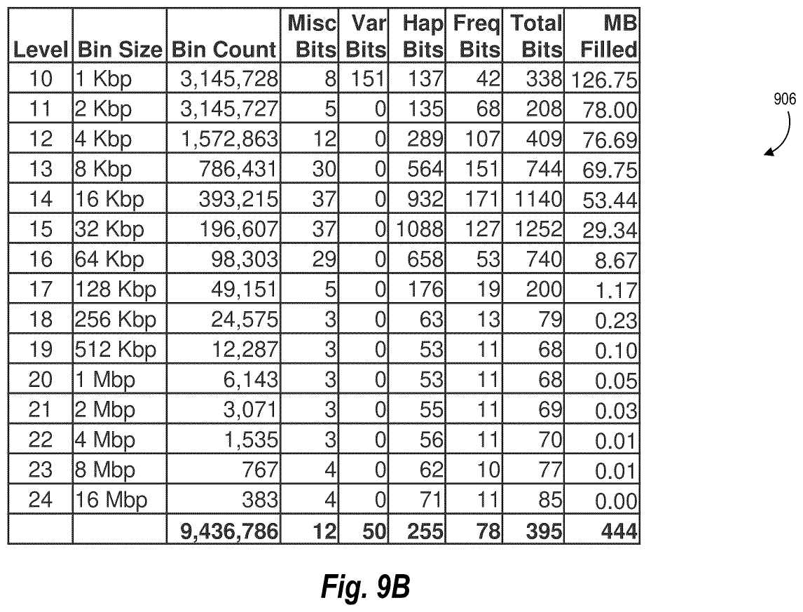

As mentioned above, in certain described embodiments, the read alignment adjustment system 106 provides improvements in efficiency and total data storage over existing systems. In particular, in certain implementations, the read alignment adjustment system 106 utilizes a haplotype data structure comprising a hierarchical partitioning of population variations relative to a primary contiguous sequence for a reference genome (e.g., as described above in relation to FIGS. 7-8). To illustrate, FIGS. 9A-9B show experimental results of the read alignment adjustment system 106 utilizing a haplotype data structure, in accordance with one or more of the disclosed embodiments, to encode population variation data for a reference genome.

For instance, FIG. 9A illustrates various measures of efficiency in bit usage by a haplotype data structure according to one embodiment, as well as an overall space comparison between the haplotype data structure and an existing augmented graph reference genome (indicated as “Est. Old-Graph Space”). As shown in table 902, for example, the haplotype data structure allocates 1.79 bits per base, fills 1.30 bits per base, and utilizes 0.37 bits per base in each bin. Also, the illustrated haplotype data structure allocates 0.53 bits per haplotype allele, fills 0.39 bits per haplotype allele, and utilizes 0.11 bits per haplotype allele in each base bin. Further, the illustrated haplotype data structure allocates 5.70 bits per alternate allele, fills 5.15 bits per alternate allele, and utilizes 1.18 bits per alternate allele in each base bin. Further, as shown in table 904, the illustrated embodiment includes a total memory allocation of 612 MB for the haplotype data structure and utilizes and additional 1009 MB for haplotype polymers, for a total memory allocation of 1.6 GB, compared to a total memory allocation of 65 GB for at least one existing augmented graph reference genome. Indeed, as illustrated by FIG. 9A, embodiments of the haplotype data structure can implement improvements to efficiency of data storage when encoding population variation relative to a reference genome.

Moreover, FIG. 9B illustrates bit allocation for multiple levels of the haplotype data structure of FIG. 9A, including an indication of a bin size (i.e., a reference span length) for bins of each respective level, bit usage at each level and overall for the various variant data encoded, and total MB of data filled at each level and within the haplotype data structure overall. As shown in table 906, for example, each successive level of the illustrated haplotype data structure occupies less memory relative to lower bins (e.g., bins spanning few nucleobase positions). Indeed, as shown in FIGS. 9A-9B, the example embodiment of the haplotype data structure implements improved efficiency and overall data storage of population variations for a reference genome in comparison with existing systems, such as existing augmented graph reference genomes.

A mentioned previously, in some embodiments, the read alignment adjustment system 106 utilizes a haplotype data structure, such as described above in relation to FIGS. 7-8, to determine alignment score adjustments for candidate alignments of nucleotide reads based on variant data encoded within the haplotype data structure. For example, FIG. 10 illustrates an overview of a series of acts 1000 for determining one or more alignment score adjustments for a candidate alignment of a nucleotide read utilizing a haplotype data structure according to one or more embodiments.

For instance, the series of acts 1000 includes an act 1002 of generating a primary alignment score for a candidate alignment of a nucleotide read from a genomic sample. As illustrated, the read alignment adjustment system 106 identifies a candidate alignment between a nucleotide read 1003 from a genomic sample with a primary contiguous sequence for a reference genome. In some embodiments, for example, the read alignment adjustment system 106 determines a set of candidate alignments for the nucleotide read 1003 (or a subset of overlapping nucleotide reads) and generates a respective set of primary alignment scores, such as described above in relation to FIGS. 3A and 5A. For each candidate alignment of the set of candidate alignments, the read alignment adjustment system 106 can perform the series of acts 1000 to determine alignment score adjustments utilizing a haplotype data structure 1005 (e.g., a haplotype data structure as described above in relation to FIGS. 7-8).

As also shown in FIG. 10, the series of acts 1000 includes an act 1004 of identifying a bin of the haplotype data structure 1005 with a corresponding reference span that includes the entirety of the nucleotide read 1003 (e.g., a bin that spans every genomic coordinate of the candidate alignment with the primary contiguous sequence). As similarly described above in relation to FIGS. 7-8, for example, the haplotype data structure 1005 comprises a base level of base-level bins comprising respective base-level reference spans corresponding to genomic regions of a first length between respective genomic coordinates of the reference genome. Further, the haplotype data structure 1005 comprises one or more successive levels of higher-level bins and offset higher level bins comprising respective higher-level reference spans corresponding to expanded genomic regions of a greater length (relative to the first length) between respective genomic coordinates of the reference genome. While FIG. 10 shows a single successive level of the haplotype data structure 1005, the haplotype data structure 1005 can include additional successive levels, such as shown in FIG. 8 (e.g., to provide sufficient bins with reference spans of adequate length to include all nucleobases of relatively longer nucleotide reads).

As illustrated, the read alignment adjustment system 106 queries the haplotype data structure 1005 to identify a base-level bin, a higher-level bin, or an offset higher-level bin with a corresponding reference span that includes the nucleotide read 1003. In the implementation shown, for example, the read alignment adjustment system 106 identifies an offset higher-level bin of the haplotype data structure 1005 that includes the entirety of the nucleotide read 1003. As also described above in relation to FIGS. 7-8, the higher-level bins and offset higher-level bins of each successive level of the haplotype data structure 1005 include variant-data indices indicating combinations of variant data from the corresponding base-level bins. Accordingly, the read alignment adjustment system 106 identifies one or more locally distinct haplotypes within the identified bin and, based on the variant-data indices, identifies variant data within the corresponding base-level bins for the respective one or more locally distinct haplotypes.

Moreover, as shown in FIG. 10, the series of acts 1000 includes an act 1006 of determining one or more alignment score adjustments based on the variant data from the identified bin of the haplotype data structure 1005. As mentioned, for example, each given base-level bin of the haplotype data structure 1005 includes variant data for locally distinct haplotypes within the respective reference span of the given base-level bin, such as allele-variant differences between the respective locally distinct haplotypes and the primary contiguous sequence (e.g., as described above in relation to FIG. 7). Further, in some embodiments, bins of the haplotype data structure 1005 also include population frequency data (e.g., population allele frequencies) for the respective locally distinct haplotypes. As also mentioned, the higher-level bins of the haplotype data structure 1005 include variant-data indices indicating combinations of the variant data of corresponding base-level bins. As shown in FIG. 10, for example, the variant data from the identified bin includes a variant data matrix 1007 representing allele-variant differences from locally distinct haplotypes and variant positions for the allele-variant differences. As indicated by the ellipsis (or dots) in FIG. 10, the read alignment adjustment system 106 can identify, determine, generate, or utilize more locally distinct haplotypes and/or alignment score adjustments than those depicted in FIG. 10.

To further illustrate, FIG. 10 shows that the variant data matrix 1007 indicates three allele-variant differences (indicated as “- T - - G A”) between a first locally distinct haplotype (indicated as “Haplotype 1”) and the primary contiguous sequence for the reference genome. Thus, by comparing the nucleobases of the nucleotide read 1003 (indicated as “A A T C G A”) with the first locally distinct haplotype, the read alignment adjustment system 106 determines a first set of alignment score adjustments including a decrease to the primary alignment score for the mismatch between adenine in the nucleotide read 1003 and thymine in the first haplotype at the second nucleobase position, and increases to the primary alignment score for the matching guanine and adenine in the nucleotide read 1003 and the first haplotype at the respective fifth and sixth nucleobase positions. Further, the variant data matrix 1007 indicates two allele-variant differences (indicated as “A - - C - -”) between a second locally distinct haplotype (indicated as “Haplotype 2”) and the primary contiguous sequence for the reference genome. Thus, by comparing the nucleobases of the nucleotide read 1003 with the second locally distinct haplotype, the read alignment adjustment system 106 determines a second set of alignment score adjustments including increases to the primary alignment score for the matching adenine and cytosine in the nucleotide read 1003 and the second haplotype at the respective first and fourth nucleobase positions.

Accordingly, as illustrated in FIG. 10, the read alignment adjustment system 106 determines alignment score adjustments for each locally distinct haplotype indicated by the identified bin based on a comparison of the nucleobases within the nucleotide read 1003 with the allele-variant difference indicated by the variant data matrix 1007 of variant data at respective nucleobase positions of the primary contiguous sequence (e.g., as further described above in relation to FIGS. 3B and 5B).

A mentioned previously, in some embodiments, the read alignment adjustment system 106 utilizes a haplotype data structure, such as described above in relation to FIGS. 7-8, to determine alignment score adjustments for candidate alignments of paired-end nucleotide reads based on variant data encoded within the haplotype data structure. For example, FIG. 11 illustrates an overview of a series of acts 1100 for determining alignment score adjustments for a candidate alignment of a paired-end nucleotide read utilizing a haplotype data structure according to one or more embodiments.

For instance, the series of acts 1100 includes an act 1102 of generating a primary alignment score for a candidate alignment of a paired-end nucleotide read from a genomic sample, the paired-end read comprising a first mate 1103a and a second mate 1103b. As illustrated, the read alignment adjustment system 106 identifies a candidate alignment between the paired nucleotide reads 1103a and 1103b from a genomic sample with a primary contiguous sequence for a reference genome. In some embodiments, for example, the read alignment adjustment system 106 determines a set of candidate alignments for the first mate 1103a and the second mate 1103b, wherein mate alignments for each of the candidate alignments are within a threshold distance of one another (e.g., as described above in relation to FIG. 4). For each candidate alignment of the mates 1103a and 1103b of the paired-end read, the read alignment adjustment system 106 generates a respective set of primary alignment scores, such as described above in relation to FIGS. 3A and 5A. For each candidate alignment of the set of candidate alignments, the read alignment adjustment system 106 can perform the series of acts 1100 to determine alignment score adjustments utilizing a haplotype data structure 1105 (e.g., a haplotype data structure as described above in relation to FIGS. 7-8).

As also shown in FIG. 11, the series of acts 1100 includes an act 1104 of identifying a bin of the haplotype data structure 1105 with a corresponding reference span that includes both mates 1103a and 1103b of the paired-end nucleotide read (e.g., a bin that spans every genomic coordinate of the candidate alignment of the paired-end read with the primary contiguous sequence). As similarly described above in relation to FIGS. 7-8 and 10, for example, the haplotype data structure 1105 comprises a base-level of base-level bins comprising respective base-level reference spans corresponding to genomic regions of a first length between respective genomic coordinates of the reference genome. Further, the haplotype data structure 1105 comprises multiple successive levels of higher-level bins and offset higher level bins comprising respective higher-level reference spans corresponding to expanded genomic regions of a greater length (relative to the first length) between respective genomic coordinates of the reference genome. While FIG. 11 shows three successive levels of the haplotype data structure 1105, the haplotype data structure 1005 can include additional successive levels (and significantly more bins than illustrated within each respective level).

As illustrated, the read alignment adjustment system 106 queries the haplotype data structure 1105 to identify a base-level bin, a higher-level bin, or an offset higher-level bin with a corresponding reference span that includes both mates 1103a and 1103b of the paired-end nucleotide read. In the implementation shown, for example, the read alignment adjustment system 106 identifies an offset higher-level bin within a third successive level of the haplotype data structure 1105 that includes both mates 1103a and 1103b of the paired-end nucleotide read. As also described above in relation to FIGS. 7-8 and 10, the higher-level bins and offset higher-level bins of each successive level of the haplotype data structure 1105 include variant-data indices indicating combinations of variant data from the corresponding base-level bins. Accordingly, the read alignment adjustment system 106 identifies one or more locally distinct haplotypes within the identified bin and, based on the variant-data indices, identifies variant data within the corresponding base-level bins for the respective one or more locally distinct haplotypes.

Moreover, as shown in FIG. 11, the series of acts 1100 includes an act 1106 of determining alignment score adjustments for the first mate 1103a and the second mate 1103b based on the variant data from the identified bin of the haplotype data structure 1105. As mentioned, for example, each given base-level bin of the haplotype data structure 1105 includes variant data for locally distinct haplotypes within the respective reference span of the given base-level bin, such as allele-variant differences between the respective locally distinct haplotypes and the primary contiguous sequence (e.g., as described above in relation to FIGS. 7 and 10). Further, in some embodiments, bins of the haplotype data structure 1105 also include population frequency data for the respective locally distinct haplotypes. As also mentioned, the higher-level bins of the haplotype data structure 1105 include variant-data indices indicating combinations of the variant data of corresponding base-level bins. As shown in FIG. 11, for example, the variant data from the identified bin includes a matrix 1107 representing allele-variant differences from locally distinct haplotypes and variant positions for the allele-variant differences.

To further illustrate, FIG. 11 shows that the variant data matrix 1107 indicates three allele-variant differences (indicated as “- T - - G A”) between a first locally distinct haplotype (indicated as “Haplotype 1”) and the primary contiguous sequence for the reference genome at nucleobase positions corresponding to the candidate alignment of the first mate 1103a of the paired-end read. Thus, by comparing the nucleobases of the first mate 1103a (indicated as “A A T C G A”) with the first locally distinct haplotype, the read alignment adjustment system 106 determines a first set of alignment score adjustments, for the first mate 1103a, including a decrease to the primary alignment score for the mismatch between adenine in the first mate 1103a and thymine in the first haplotype at the second nucleobase position of the first mate 1103a, and increases to the primary alignment score for the matching guanine and adenine in the first mate 1103a and the first haplotype at the respective fifth and sixth nucleobase positions of the first mate 1103a.

Also, the variant data matrix 1107 indicates one allele-variant difference (indicated as “- - - T - -”) between the first locally distinct haplotype (indicated as “Haplotype 1”) and the primary contiguous sequence for the reference genome at nucleobase positions corresponding to the candidate alignment of the second mate 1103b of the paired-end read. Thus, by comparing the nucleobases of the second mate 1103b (indicated as “C C G T A C”) with the first locally distinct haplotype, the read alignment adjustment system 106 determines a first set of alignment score adjustments, for the second mate 1103b, including an increase to the primary alignment score for the matching thymine in the second mate 1103b and the first haplotype at the fourth nucleobase position of the second mate 1103b.

Further, the variant data matrix 1107 indicates two allele-variant differences (indicated as “A - - C - -”) between a second locally distinct haplotype (indicated as “Haplotype 2”) and the primary contiguous sequence for the reference genome at nucleobase positions corresponding to the candidate alignment of the first mate 1103a of the paired-end nucleotide reads. Thus, by comparing the nucleobases of the first mate 1103a of the paired-end nucleotide reads with the second locally distinct haplotype, the read alignment adjustment system 106 determines a second set of alignment score adjustments, for the first mate 1103a, including increases to the primary alignment score for the matching adenine and cytosine in the first mate 1103a and the second haplotype at the respective first and fourth nucleobase positions of the first mate 1103a.

Also, the variant data matrix 1107 indicates two allele-variant differences (indicated as “G - - T - -”) between the second locally distinct haplotype (indicated as “Haplotype 2”) and the primary contiguous sequence for the reference genome at nucleobase positions corresponding to the candidate alignment of the second mate 1103b of the paired-end read. Thus, by comparing the nucleobases of the second mate 1103b of the paired-end nucleotide read with the second locally distinct haplotype, the read alignment adjustment system 106 determines a second set of alignment score adjustments, for the second mate 1103b, including a decrease to the primary alignment score for the mismatch between cytosine in the second mate 1103b and guanine in the second haplotype at the first nucleobase position of the second mate 1103b, and an increase to the primary alignment score for the matching thymine in the second mate 1103b and the second haplotype at the fourth nucleobase position of the second mate 1103b.

Accordingly, as illustrated in FIG. 11, the read alignment adjustment system 106 determines alignment score adjustments for each locally distinct haplotype indicated by the identified bin based on a comparison of the nucleobases within the first mate 1103a and the second mate 1103b of the nucleotide read with the allele-variant difference indicated by the matrix 1107 of variant data at respective nucleobase positions of the primary contiguous sequence (e.g., as further described above in relation to FIGS. 3B, 5B, and 10).

Additionally, as shown in FIG. 11, the series of acts 1100 includes an act 1108 of summing the alignment score adjustments corresponding to the first mate 1103a and the second mate 1103b of the paired-end read for each respective locally distinct haplotype. In some embodiments, for example, the read alignment adjustment system 106 sums, for each locally distinct population haplotype indicated within the bin identified by act 1104, the alignment score adjustments for the first mate 1103a and the alignment score adjustments for the second mate 1103b to determine adjusted alignment scores for the paired-end read relative to each identified locally distinct haplotype. Moreover, in one or more embodiments, the read alignment adjustment system 106 selects predicted alignments of the first and second mates of a paired-end read with the primary contiguous sequence of with a locally distinct population haplotype based on a highest sum of adjusted alignment scores corresponding to each candidate alignment within a set of candidate alignments for the paired-end read.

Furthermore, in some embodiments, the read alignment adjustment system 106 utilizes a haplotype data structure, such as described above in relation to FIGS. 7-8, to determine alignment score adjustments for candidate alignments of other types of nucleotide reads, such as transcriptomic reads representing spliced RNA sequences, based on variant data encoded within the haplotype data structure. For example, FIG. 12 illustrates an example implementation of the read alignment adjustment system 106 utilizing a haplotype data structure 1200 to determine alignment score adjustments for an RNA spliced alignment 1202 of a transcriptomic read according to one or more embodiments.

As shown in FIG. 12, the RNA spliced alignment 1202 comprises a first candidate read alignment 1204a of approximately 50 nucleobases, a first spliced sequence 1206a of approximately 11,250 nucleobases; a second candidate read alignment 1204b of approximately 50 nucleobases; a second spliced sequence 1206b of approximately 13,450 nucleobases; and a third candidate read alignment 1204c of approximately 50 nucleobases. As illustrated, the read alignment adjustment system 106 identifies the shortest bin within the haplotype data structure 1200 (shown as bin number 19 on level 15 in FIG. 12) in which a full RNA spliced alignment (e.g., the RNA spliced alignment 1202) fits according to an initial alignment of the RNA spliced alignment 1202 with a primary contiguous sequence for a reference genome-whether that shortest bin be a base-level bin, a higher-level bin, or an offset higher-level bin within the haplotype data structure 1200. Accordingly, the read alignment adjustment system 106 can determine alignment score adjustments for the RNA spliced alignment 1202 in relation to one or more locally distinct haplotypes identified within the selected bin (shown as bin number 19 on level 15 in FIG. 12).

As further shown in FIG. 12, the first candidate read alignment 1204a of the RNA spliced alignment 1202 includes nucleobase positions spanning two consecutive base-level bins (bin number 1 and bin number 2 in FIG. 12). Thus, as illustrated in FIG. 12, the read alignment adjustment system 106 first identifies variant data (e.g., allele-variant differences between the primary contiguous sequence and locally distinct population haplotypes within the respective bin) within the first identified bin (shown as bin number 1). Then, the read alignment adjustment system 106 identifies variant-data indices within the corresponding bin on the successive level (shown as bin number 2), followed by the corresponding bin on the next successive level (shown as bin number 3), to adjust alignment scores according to the locally distinct haplotypes at each respective level. Proceeding to the next base-level bin covering nucleobases of the first candidate read alignment 1204a (bin number 2), the read alignment adjustment system 106 further adjusts alignment scores for variant data within that bin (bin number 2) and locally distinct haplotypes identified by variant-data indices within corresponding bins at each successive level (bin number 5 and bin number 6). Having identified and adjusted alignment scores according to variant data and variant-data indices within bin number 1 through bin number 6, the read alignment adjustment system 106 identifies variant-data indices within a corresponding bin on the next successive level (bin number 7).

Moreover, following a similar process to determine alignment score adjustments for the second candidate read alignment 1204b of the RNA spliced alignment 1202, the read alignment adjustment system 106 identifies and adjusts for variant data and variant-data indices within bin number 8 through bin number 12 shown in FIG. 12. By identifying variant-data indices within a bin of the next successive level (bin number 13) corresponding to bin number 7 and bin number 12, the read alignment adjustment system 106 determines further alignment score adjustments according to the locally distinct haplotypes identified within bin number 13. Subsequently, following a similar process for the third candidate read alignment 1204c of the RNA spliced alignment 1202, the read alignment adjustment system 106 identifies and adjusts for variant data and variant-data indices within bin number 15 through bin number 18. Note that, according to the initial alignment, the third candidate read alignment 1204c falls entirely within a single base-level bin (bin 14). Finally, the read alignment adjustment system 106 identifies variant-data indices within the higher-level bin (bin number 19) corresponding to a complete RNA spliced alignment (e.g., the RNA spliced alignment 1202) and determines one or more final alignment score adjustments in relation to the one or more locally distinct haplotypes identified within the respective bin.

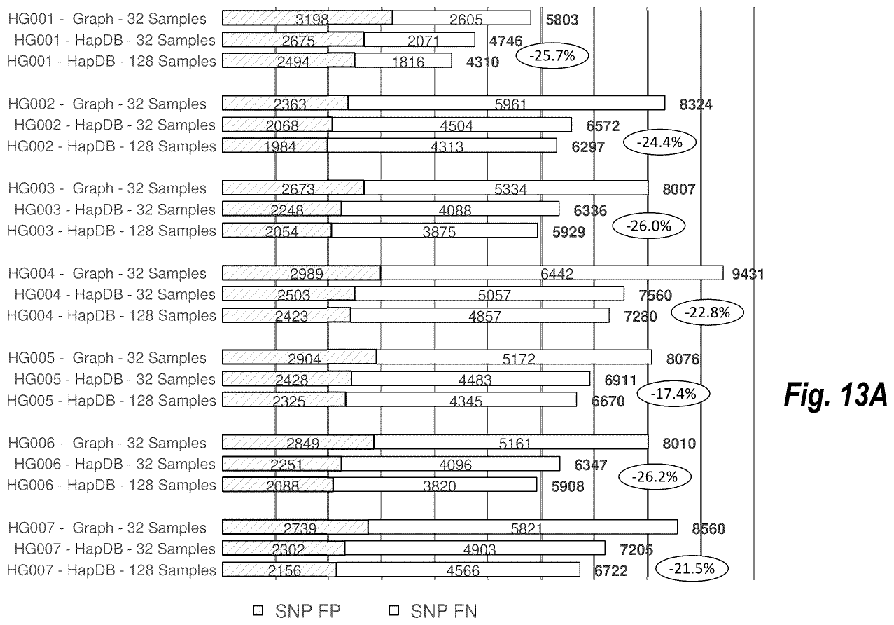

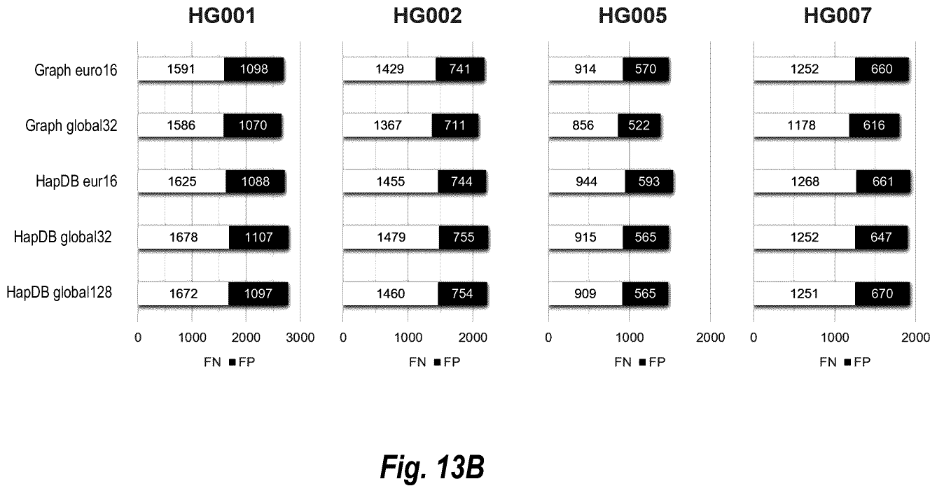

As mentioned above, in certain described embodiments, the read alignment adjustment system 106 implements efficient and accurate mapping of alignment of nucleotide reads from a genomic sample with genomic regions of a reference genome. To illustrate, FIGS. 13A-13B show experimental results of the read alignment adjustment system 106 utilizing a haplotype data structure, in accordance with one or more of the disclosed embodiments, to determine predicted alignments of nucleotide reads. In particular, FIG. 13A illustrates comparative experimental results of identifying single nucleotide polymorphisms (SNPs) based on read alignments generated according to one or more embodiments, and FIG. 13B illustrates comparative experimental results of identifying insertions or deletions (indels) based on read alignments generated according to one or more embodiments.

As mentioned, FIG. 13A provides comparative experimental results of identifying single nucleotide polymorphisms (SNPs) based on read alignments generated according to one or more embodiments and read alignments generated utilizing existing sequencing systems. In particular, FIG. 13A includes a table of experimental results of identifying SNPs in read aligned by existing sequencing systems and the read alignment adjustment system 106, as reflected by false positives (SNP FP) false negatives (SNP FN), wherein each respective set of three rows corresponds to a standard reference genomic sample having identified ground-truth variants. Specifically, the ground-truth datasets utilized to generate the provided experimental results include seven human reference genome samples-Genome in a Bottle (GIAB) samples HG001, HG002, HG003, HG004, HG005, and HG007, respectively, having corresponding ground-truth variant calls. Moreover, each row of the illustrated table provides experimental results of identifying SNPs within the respective reference sample datasets, as reflected by a number of false negatives (FN) and/or false positives (FP). Specifically, the first row of each respective set of three rows provides experimental results of an existing sequencing system utilizing an augmented graph reference genome, whereas the second and third rows of each respective set of three rows provide experimental results of two respective implementations of the read alignment adjustment system 106 utilizing embodiments of the haplotype data structure. Also, each set of three rows includes an indication of the percentage increase in accuracy between the implementations represented by the respective first and third rows.

Indeed, as illustrated in FIG. 13A, the read alignment adjustment system 106 can efficiently predict read alignments for nucleotide reads from a genomic sample with improved accuracy in identifying SNPs relative to existing sequencing systems, as indicated by the comparative number of false positives (FPs) and false negatives (FNs) identified within the provided experimental results.

Further, FIG. 13B provides comparative experimental results of identifying insertions or deletions (indels) based on read alignments generated according to one or more embodiments. In particular, FIG. 13B includes a table of experimental results, wherein the first two rows correspond to existing sequencing systems utilizing augmented graph reference genomes for mapping and alignment, and wherein the final three rows correspond to exemplary implementations of the read alignment adjustment system 106 utilizing embodiments of the haplotype data structure for mapping and alignment. Also, each column of the table of experimental results corresponds to a standard reference genomic sample—Genome in a Bottle (GIAB) samples HG001, HG002, HG005, and HG007, respectively, having corresponding ground-truth variant calls. Specifically, the row of results indicated as “Graph euro 16” includes experimental results of an existing sequencing system utilizing an augmented graph reference genome comprising 16 haplotypes derived from a European population sample, whereas the row or results indicated as “Graph global32” includes experimental results of an existing sequencing system utilizing an augmented graph reference genome comprising 32 haplotypes derived from a global population sample. Moreover, the rows of results indicated as “HapDB eur16,” “HapDB global32,” and “HapDB global128” include experimental results of implementations of the read alignment adjustment system 106 utilizing haplotype data structures comprising 16 European haplotypes, 32 global haplotypes, and 128 global haplotypes, respectively.

Indeed, as shown in FIG. 13B, the read alignment adjustment system 106 can efficiently predict read alignments for nucleotide reads from a genomic sample with comparably accurate results in identifying indels relative to existing sequencing systems, as indicated by the comparative number of false positives (FPs) and false negatives (FNs) identified within the provided experimental results. Also, as shown by the provided experimental results of FIG. 13B, the read alignment adjustment system 106 can provide further improvements in accuracy of identifying indels within a genomic sample as a greater number of haplotypes are implemented within the haplotype data structure (a capability often unachievable by existing sequencing systems as augmented graph reference genomes become unreasonably large).

As mentioned above, in some embodiments, the read alignment adjustment system 106 aligns and determines adjusted alignment scores for a genomic sample's nucleotide reads utilizing an improved haplotype data structure encoding allele-variant differences between a primary contiguous sequence and population haplotypes across a linear reference genome. By contrast, some existing sequencing systems aligns and determine alignment scores for a genomic sample's nucleotide reads utilizing a graph reference genomes including both a linear reference genome and graph augmentations representing alternate contiguous sequences. To further illustrate the different approaches and corresponding computing-efficiency savings, FIG. 14A depicts an example of an existing sequencing system aligning a nucleotide read of a genomic sample with a graph reference genome, and FIG. 14B depicts an example implementation of the read alignment adjustment system 106 initially aligning the same nucleotide read of the genomic sample with a primary contiguous sequence (or other reference sequence) and subsequently determining alignment score adjustments for the initial alignments in relation to population haplotypes encoded within an haplotype data structure according to one or more embodiments.

As shown in FIG. 14A, the existing sequencing system aligns a nucleotide read (shown as “Read”) of a genomic sample with each of a linear reference sequence (shown as “Ref”) of a graph reference genome and three alternate contiguous sequences (shown as “Alt1,” “Alt2,” and “Alt3”) of the graph reference genome. As suggested by FIG. 14A, the existing sequencing system must not only store in memory the linear reference sequence and alternate contiguous sequences as part of the graph reference genome but must also determine individual alignment scores for candidate alignments between the nucleotide read and each of the linear reference sequence and alternate contiguous sequences.

In particular, as shown in FIG. 14A, the existing sequencing system determines alignment scores of 135, 135, 140, and 145 for a candidate alignment of the nucleotide read respectively with the linear reference sequence (shown as “Ref”), a first alternate contiguous sequence (shown as “Alt1”), a second alternate contiguous sequence (shown as “Alt2”), and a third alternate contiguous sequence (“Alt3”). The existing sequencing system determines such alignment scores in part by accounting for mismatches (marked by “X” in FIG. 14A) between the nucleotide read and the linear reference sequence or the three different alternate contiguous sequences-including a mismatch caused by a sequencing error (identified as “error” in FIG. 14A). Only after individually scoring candidate alignments with each of the linear reference sequence and the three different alternate contiguous sequences does the existing sequencing system identify a candidate alignment between the nucleotide read and the third alternate contiguous sequence (shown as “Alt3”) as a highest (maximum) alignment score of the various candidate read alignments.

In contrast, as shown in FIG. 14B, the read alignment adjustment system 106 aligns the nucleotide read (shown as “Read”) of the genomic sample with a primary contiguous sequence or other reference sequence (shown as “Ref”) and determines a primary alignment score of 135 for a candidate alignment between the nucleotide read and the reference sequence. Indeed, the read alignment adjustment system 106 performs a single alignment operation for the nucleotide read in FIG. 14B instead of four separate alignment operations for the nucleotide read in FIG. 14A. As further indicated by FIG. 14B, instead of individually storing and scoring alternative contiguous sequences, the read alignment adjustment system 106 adjusts the primary alignment score (e.g., by “+5” or “−5”) based on comparing the nucleotide read with allele-variant differences from locally distinct population haplotypes (shown as “Hap1,” “Hap2,” and “Hap3”), where the allele-variant differences are encoded for reference spans within a haplotype data structure. In particular, the read alignment adjustment system 106 (i) adjusts the primary alignment score up and down (shown as “+5” and “−5”) to an adjusted alignment score of 135 to account for allele-variant differences from a first locally distinct population haplotype (shown as “Hap1”), (ii) increases the primary alignment score (shown as “+5”) to an adjusted alignment score of 140 to account for allele-variant differences from a second locally distinct population haplotype (shown as “Hap2”), and (iii) increases the primary alignment score (shown as “+5” and “+5”) to an adjusted alignment score of 145 to account for allele-variant differences from a third locally distinct population haplotype (shown as “Hap3”).

As further shown in FIG. 14B, the read alignment adjustment system 106 further (a) converts the adjusted alignment scores to alignment likelihoods, (b) adjusts the alignment likelihoods based on corresponding allele frequencies to generate adjusted alignment likelihoods, and (c) converts a weighted sum of the adjusted alignment likelihoods to a replacement alignment score for a candidate alignment corresponding to a location of the primary contiguous sequence. In particular, the read alignment adjustment system 106 converts the adjusted alignment score of 135 to a first alignment likelihood (shown as “Lik1”) and adjusts the first alignment likelihood based on a corresponding haplotype frequency for a particular combination of alleles (shown as “Freq1”) to generate a first adjusted alignment likelihood (not shown). The read alignment adjustment system 106 also converts the adjusted alignment score of 140 to a second alignment likelihood (shown as “Lik2”) and adjusts the second alignment likelihood based on a corresponding haplotype frequency for a particular combination of alleles (shown as “Freq2”) to generate a second adjusted alignment likelihood (not shown). The read alignment adjustment system 106 likewise converts the adjusted alignment score of 145 to a third alignment likelihood (shown as “Lik3”) and adjusts the third alignment likelihood based on a corresponding haplotype frequency for a particular combination of alleles (shown as “Freq3”) to generate a third adjusted alignment likelihood (not shown). The read alignment adjustment system 106 further determines a weighted sum (logarithmic) of the first, second, and third adjusted alignment likelihoods to generate a replacement alignment score or a final adjusted alignment score (shown as “Adj Score” in FIG. 14B) for a particular candidate alignment of the nucleotide read with the primary contiguous sequence or other reference sequence (shown as “Ref”). As indicated above, the terms replacement alignment score and final adjusted alignment score are used interchangeably.

As indicated by a comparison of FIG. 14A and FIG. 14B, the existing sequencing system determines a highest alignment score of 145 for a candidate alignment between the nucleotide read and the third alternate contiguous sequence (shown as “Alt3”), and the read alignment adjustment system 106 determines a replacement alignment score of around 145 for the candidate alignment of the nucleotide read with the primary contiguous sequence with adjustments accounting for allele-variant difference of a third locally distinct population haplotype. But the read alignment adjustment system 106 arrives at the very similar alignment score with better computing efficiency by avoiding the computationally heavy operations of multiple alignments and full alignment scoring.

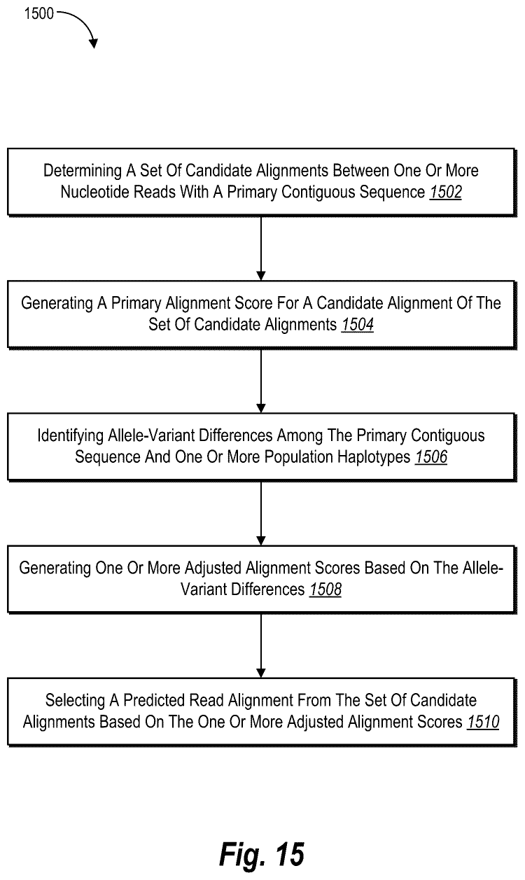

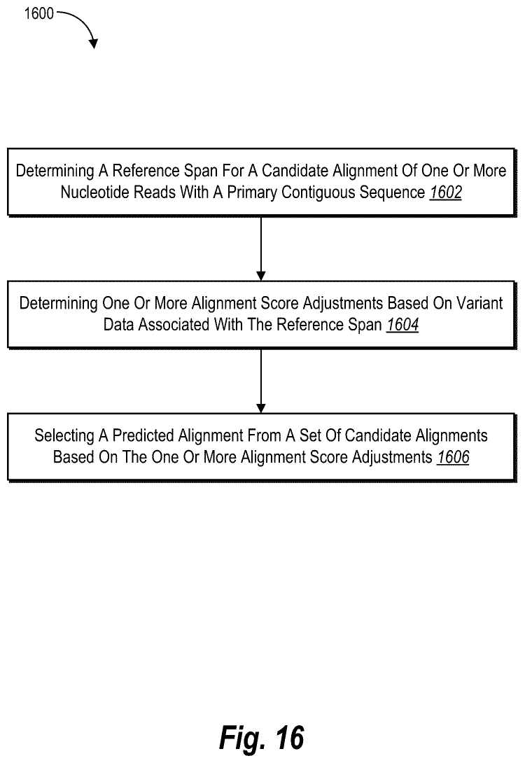

Turning now to FIGS. 15-16, these figures illustrate two example flowcharts of two respective series of acts for determining a predicted read alignment for one or more nucleotide reads from a genomic sample in accordance with one or more embodiments. While FIGS. 15-16 illustrate acts according to particular embodiments, alternative embodiments may omit, add to, reorder, and/or modify any of the acts shown in FIGS. 15-16. The acts of FIGS. 15 and/or 16 can be performed as part of a method. Alternatively, a non-transitory computer readable storage medium can comprise instructions that, when executed by one or more processors, cause a computing device to perform the acts depicted in FIGS. 15 and/or 16. In still further embodiments, a system comprising at least one processor and a non-transitory computer readable medium comprising instructions that, when executed by one or more processors, cause the system to perform the acts of FIGS. 15 and/or 16.

As shown in FIG. 15, the series of acts 1500 includes an act 1502 of determining a set of candidate alignments between one or more nucleotide reads with a primary contiguous sequence, an act 1504 of generating a primary alignment score for a candidate alignment of the set of candidate alignments, and act 1506 of identifying allele-variant differences among the primary contiguous sequence and one or more population haplotypes, an act 1508 of generating one or more adjusted alignment scores based on the allele-variant differences, and an act 1510 of selecting a predicted read alignment from the set of candidate alignments based on the one or more adjusted alignment scores.

As shown in FIG. 16, the series of acts 1600 includes and act 1602 of determining a reference span for a candidate alignment of one or more nucleotide reads with a primary contiguous sequence, and act 1604 of determining one or more alignment score adjustments based on variant data associated with the reference span, and an act 1606 of selecting a predicted alignment from a set of candidate alignments based on the one or more alignment score adjustments.

For example, the series of acts 1500 and/or the series of acts 1600 can include acts to perform any of the operations described in the following clauses:

CLAUSE 1. A computer-implemented method comprising:
determining a set of candidate alignments between one or more nucleotide reads from a genomic sample with a primary contiguous sequence at a respective set of genomic regions of a reference genome;
generating a primary alignment score for a candidate alignment from the set of candidate alignments;
identifying one or more allele-variant differences among the primary contiguous sequence and one or more population haplotypes corresponding to a respective genomic region for the candidate alignment;
generating one or more adjusted alignment scores from the primary alignment score based on comparing the one or more nucleotide reads with the one or more allele-variant differences; and
selecting, from the set of candidate alignments, a predicted read alignment of the one or more nucleotide reads with the primary contiguous sequence or with a population haplotype from the one or more population haplotypes based on the one or more adjusted alignment scores.

CLAUSE 2. The computer-implemented method of clause 1, further comprising:
generating a replacement alignment score for the candidate alignment based on the primary alignment score and the one or more adjusted alignment scores;
generating additional replacement alignment scores for additional candidate alignments of the set of candidate alignments; and
selecting the predicted read alignment of the one or more nucleotide reads based on comparing the replacement alignment score with one or more primary alignment scores for one or more candidate alignments with one or more primary contiguous sequences and with the additional replacement alignment scores for the additional candidate alignments of the set of candidate alignments.

CLAUSE 3. The computer-implemented method of any of clauses 1-2, further comprising:
determining, for a paired-end read of the one or more nucleotide reads, that a first candidate alignment of a first mate of the paired-end read with the primary contiguous sequence is not within a threshold number of nucleobases from a second candidate alignment of a second mate of the paired-end read with the primary contiguous sequence; and
based on the first candidate alignment not being within the threshold number of nucleobases from the second candidate alignment, identifying the second candidate alignment of the second mate within a predetermined search region relative to the first candidate alignment of the first mate.

CLAUSE 4. The computer-implemented method of any of clauses 1-3, further comprising identifying the one or more allele-variant differences by querying a haplotype data structure comprising a set of bins corresponding to a set of reference spans of nucleobases from a reference genome.

CLAUSE 5. The computer-implemented method of clause 4, further comprising:
querying the haplotype data structure by identifying a reference span of the set of reference spans that includes an entire candidate alignment of the one or more nucleotide reads; and
identifying the one or more allele-variant differences stored within a bin of the set of bins corresponding to the identified reference span.

CLAUSE 6. The computer-implemented method of clause 5, further comprising identifying the one or more allele-variant differences stored within the bin corresponding to the identified reference span by comparing the one or more nucleotide reads with allele-variant differences stored within the bin from one or more locally distinct population haplotype sequences.

CLAUSE 7. The computer-implemented method of any of clauses 1-6, further comprising:
querying, for a first mate and a second mate of a paired-end read of the one or more nucleotide reads, a haplotype data structure by identifying a reference span of a set of reference spans that includes a first candidate alignment of the first mate and a second candidate alignment of the second mate;
generating, for each locally distinct population haplotype encoded by the reference span, a first adjusted alignment score for the first mate and a second adjusted alignment score for the second mate based on comparing the first mate and the second mate with the one or more allele-variant differences stored within a bin of a set of bins corresponding to the identified reference span;
summing, for each locally distinct population haplotype encoded by the reference span, the first adjusted alignment score for the first mate and the second adjusted alignment score for the second mate; and
selecting, from the set of candidate alignments, a first predicted alignment of the first mate and a second predicted alignment of the second mate with the primary contiguous sequence or with a locally distinct population haplotype based on a highest sum of adjusted alignment scores.

CLAUSE 8. The computer-implemented method of clause 7, further comprising:
generating a summed replacement alignment score for a subset of candidate alignments for the first mate and the second mate based on the primary alignment score and the first adjusted alignment score and the second adjusted alignment score for each locally distinct population haplotype encoded by the reference span;
generating additional summed replacement alignment scores for additional subsets of candidate alignments of the set of candidate alignments for the first mate and the second mate; and
selecting, from the set of candidate alignments, the first predicted alignment and the second predicted alignment based on comparing the summed replacement alignment score with one or more primary alignment scores for one or more candidate alignments with one or more primary contiguous sequences and with the additional summed replacement alignment scores for the additional subsets of candidate alignments of the set of candidate alignments.

CLAUSE 9. The computer-implemented method of any of clauses 1-8, further comprising generating the one or more adjusted alignment scores without comparing nucleobases of the one or more nucleotide reads with nucleobases of the one or more population haplotypes at base positions where there are no allele-variant differences.

CLAUSE 10. The computer-implemented method of any of clauses 1-9, further comprising identifying the one or more allele-variant differences by comparing nucleobases within the one or more nucleotide reads with data representing one or more single nucleotide polymorphisms (SNPs) within the one or more population haplotypes corresponding to the respective genomic region.

CLAUSE 11. The computer-implemented method of any of clauses 1-10, further comprising identifying the one or more allele-variant differences by comparing the one or more nucleotide reads with data representing one or more insertions or deletions (indels) within the one or more population haplotypes corresponding to the respective genomic region.

CLAUSE 12. The computer-implemented method of any of clauses 1-11, further comprising generating at least one adjusted alignment score of the one or more adjusted alignment scores from the primary alignment score by:
determining that the one or more nucleotide reads comprise one or more haplotype nucleotide variants of a locally distinct population haplotype that differ from the primary contiguous sequence in the respective genomic region; and
increasing, based on the one or more nucleotide reads comprising the one or more haplotype nucleotide variants, the primary alignment score to generate the at least one adjusted alignment score.

CLAUSE 13. The computer-implemented method of any of clauses 1-12, further comprising generating at least one adjusted alignment score of the one or more adjusted alignment scores from the primary alignment score by:
determining that the one or more nucleotide reads comprise one or more reference nucleobases of the primary contiguous sequence that differ from a locally distinct population haplotype in the respective genomic region; and
decreasing, based on the one or more nucleotide reads comprising one or more reference nucleobases, the primary alignment score to generate the at least one adjusted alignment score.

CLAUSE 14. The computer-implemented method of any of clauses 1-13, further comprising:
generating the one or more adjusted alignment scores by generating a set of adjusted alignment scores for a respective set of locally distinct population haplotypes corresponding to the respective genomic region of the candidate alignment;
selecting, as a replacement alignment score for the candidate alignment, a highest adjusted alignment score from the set of adjusted alignment scores; and
selecting the predicted read alignment from the set of candidate alignments based on the replacement alignment score.

CLAUSE 15. The computer-implemented method of any of clauses 1-14, further comprising:
generating the one or more adjusted alignment scores by generating a set of adjusted alignment scores for a respective set of locally distinct population haplotypes corresponding to the respective genomic region of the candidate alignment;
converting the set of adjusted alignment scores to a set of alignment likelihoods;
adjusting the set of alignment likelihoods based on corresponding allele frequencies to generate a set of adjusted alignment likelihoods;
converting a summation of the set of adjusted alignment likelihoods to a replacement alignment score for the candidate alignment; and
selecting the predicted read alignment from the set of candidate alignments based on the replacement alignment score.

CLAUSE 16. The computer-implemented method of any of clauses 1-15, further comprising adjusting at least one of the one or more adjusted alignment scores based on a population allele frequency of a population haplotype within a sample population.

CLAUSE 17. The computer-implemented method of any of clauses 1-16, further comprising generating the primary alignment score for the candidate alignment based on a given candidate alignment between the one or more nucleotide reads and a modified version of the primary contiguous sequence comprising one or more multi-base codes representing one or more single nucleotide polymorphisms (SNPs) or representing one or more insertions or deletions (indels).

CLAUSE 18. A haplotype data structure comprising:
(a) a base level having a set of base-level bins comprising:
   a set of base-level reference spans of a primary contiguous sequence for a reference genome, each base-level reference span comprising a genomic region of a first length between respective genomic coordinates of the reference genome; and
   variant data for nucleotide variants from respective sets of locally distinct population haplotypes, each locally distinct haplotype comprising a unique set of one or more allele-variant differences relative to other population haplotypes within the genomic region of a respective base-level reference span; and
(b) a successive level having a set of higher-level bins comprising:
   a set of higher-level reference spans of the primary contiguous sequence, each higher-level reference span comprising an expanded genomic region of a second length between respective genomic coordinates of the reference genome, the second length longer than the first length; and
   variant-data indices referencing combinations of the variant data from corresponding base-level bins of the set of base-level bins.

CLAUSE 19. The haplotype data structure of clause 18, wherein the variant data of the set of base-level bins includes data indications of single-nucleotide polymorphisms (SNPs) and insertions or deletions (indels) at respective genomic coordinates of the primary contiguous sequence.

CLAUSE 20. The haplotype data structure of any of clauses 18-19, wherein the set of base-level bins includes the variant data for nucleotide variants without including reference nucleobases of the primary contiguous sequence.

CLAUSE 21. The haplotype data structure of any of clauses 18-20, wherein population haplotypes having identical nucleotide variants within a given base-level bin are encoded as one locally distinct population haplotype within the given base-level bin.

CLAUSE 22. The haplotype data structure of any of clauses 18-21, wherein each base-level bin of the set of base-level bins comprises a matrix including corresponding variant data representing allele-variant differences from locally distinct haplotypes and variant positions for the allele-variant differences.

CLAUSE 23. The haplotype data structure of any of clauses 18-22, wherein each respective expanded genomic region of the set of higher-level reference spans corresponds to a consecutive pair of respective genomic regions of consecutive base-level reference spans of the set of base-level reference spans.

CLAUSE 24. The haplotype data structure of any of clauses 18-23, wherein the successive level of the haplotype data structure further comprises a set of offset higher-level bins comprising:
a set of offset higher-level reference spans of the primary contiguous sequence, each offset higher-level reference span comprising an offset expanded genomic region of the second length between respective genomic coordinates of the reference genome,
wherein the offset expanded genomic region corresponds to a consecutive pair of respective genomic regions of the set of base-level reference spans, and
wherein the set of offset higher-level reference spans are offset from the set of higher-level reference spans by one base-level reference span of the set of base-level reference spans.

CLAUSE 25. The haplotype data structure of clause 24, further comprising:
at least one additional successive level having an additional set of higher-level reference bins comprising:
   a set of additional higher-level reference spans of the primary contiguous sequence, each higher-level reference span comprising a further expanded genomic region of a third length between respective genomic coordinates of the reference genome, the third length longer than the second length; and
   variant-data indices referencing combinations of the variant data from corresponding base-level bins of the set of base-level bins.

CLAUSE 26. A computer-implemented method implementing the haplotype data structure of any of clauses 18-25, the computer-implemented method comprising:
determining, for a candidate alignment from a set of candidate alignments between one or more nucleotide reads from a genomic sample with the primary contiguous sequence, a base-level reference span of the set of base-level reference spans that includes the one or more nucleotide reads;
determining, based on variant data from a base-level bin of the set of base-level bins corresponding to the base-level reference span, one or more alignment score adjustments corresponding to one or more locally distinct haplotypes within a respective genomic region of the base-level reference span; and
selecting, from the set of candidate alignments, a predicted alignment of the one or more nucleotide reads with the primary contiguous sequence or with a population haplotype based on the one or more alignment score adjustments.

CLAUSE 27. The computer-implemented method of clause 26, further comprising:
generating a replacement alignment score for the candidate alignment based on the one or more alignment score adjustments;
generating additional replacement alignment scores for additional candidate alignments of the set of candidate alignments; and
selecting the predicted read alignment of the one or more nucleotide reads based on comparing the replacement alignment score with the additional replacement alignment scores.

CLAUSE 28. The computer-implemented method of clause 27, further comprising:
determining, for a candidate alignment from a set of candidate alignments between one or more nucleotide reads from a genomic sample with the primary contiguous sequence, a higher-level reference span of the set of higher-level reference spans that includes an entire candidate alignment of the one or more nucleotide reads;
determining, from variant-data indices of a higher-level bin of the set of higher-level bins corresponding to the higher-level reference span, a subset of locally distinct population haplotypes within a respective expanded genomic region of the higher-level reference span;
determining, from variant data of a first base-level bin of the set of base-level bins corresponding to a first respective genomic region within the respective expanded genomic region, a first set of alignment-score adjustments for one or more respective locally distinct population haplotypes of the subset of locally distinct population haplotypes;
determining, from variant data of a second base-level bin of the set of base-level bins corresponding to a second respective genomic region within the respective expanded genomic region, a second set of alignment-score adjustments for one or more respective locally distinct population haplotypes of the subset of locally distinct population haplotypes; and
selecting, from the set of candidate alignments, a predicted alignment of the one or more nucleotide reads with the primary contiguous sequence or with a population haplotype based on a combination of the first set of alignment-score adjustments and the second set of alignment-score adjustments.

CLAUSE 29. A computer-implemented method implementing the haplotype data structure of any of clauses 18-25, the computer-implemented method comprising:
determining, for a candidate alignment from a set of candidate alignments between one or more nucleotide reads from a genomic sample with the primary contiguous sequence, a reference span that includes an entire candidate alignment of the one or more nucleotide reads, the reference span being selected from a lowest level of the haplotype data structure in which the one or more nucleotide reads are included in a single reference span of the set of base-level reference spans or the set of higher-level reference spans;
determining, based on variant data from one or more bins of the set of base-level bins corresponding to the reference span, one or more alignment score adjustments corresponding to one or more locally distinct haplotypes within a respective genomic region of the reference span; and
selecting, from the set of candidate alignments, a predicted alignment of the one or more nucleotide reads with the primary contiguous sequence or with a population haplotype based on the one or more alignment score adjustments.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference. The sequencing system described above sequences nucleic acid polymers present in samples received by a sequencing device, as described further above.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the read alignment adjustment system 106 can include software, hardware, or both. For example, the components of the read alignment adjustment system 106 can include one or more instructions stored on a computer-readable storage medium and executable by processors of one or more computing devices (e.g., the client device 114). When executed by the one or more processors, the computer-executable instructions of the read alignment adjustment system 106 can cause the computing devices to perform the bubble detection methods described herein. Alternatively, the components of the read alignment adjustment system 106 can comprise hardware, such as special purpose processing devices to perform a certain function or group of functions. Additionally, or alternatively, the components of the read alignment adjustment system 106 can include a combination of computer-executable instructions and hardware.

Furthermore, the components of the read alignment adjustment system 106 performing the functions described herein with respect to the read alignment adjustment system 106 may, for example, be implemented as part of a stand-alone application, as a module of an application, as a plug-in for applications, as a library function or functions that may be called by other applications, and/or as a cloud-computing model. Thus, components of the read alignment adjustment system 106 may be implemented as part of a stand-alone application on a personal computing device or a mobile device. Additionally, or alternatively, the components of the read alignment adjustment system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 17 illustrates a block diagram of a computing device 1700 that may be configured to perform one or more of the processes described above. One will appreciate that one or more computing devices such as the computing device 1700 may implement the read alignment adjustment system 106 and the sequencing system 104. As shown by FIG. 17, the computing device 1700 can comprise a processor 1702, a memory 1704, a storage device 1706, an I/O interface 1708, and a communication interface 1710, which may be communicatively coupled by way of a communication infrastructure 1712. In certain embodiments, the computing device 1700 can include fewer or more components than those shown in FIG. 17. The following paragraphs describe components of the computing device 1700 shown in FIG. 17 in additional detail.

In one or more embodiments, the processor 1702 includes hardware for executing instructions, such as those making up a computer program. As an example, and not by way of limitation, to execute instructions for dynamically modifying workflows, the processor 1702 may retrieve (or fetch) the instructions from an internal register, an internal cache, the memory 1704, or the storage device 1706 and decode and execute them. The memory 1704 may be a volatile or non-volatile memory used for storing data, metadata, and programs for execution by the processor(s). The storage device 1706 includes storage, such as a hard disk, flash disk drive, or other digital storage device, for storing data or instructions for performing the methods described herein.

The I/O interface 1708 allows a user to provide input to, receive output from, and otherwise transfer data to and receive data from computing device 1700. The I/O interface 1708 may include a mouse, a keypad or a keyboard, a touch screen, a camera, an optical scanner, network interface, modem, other known I/O devices or a combination of such I/O interfaces. The I/O interface 1708 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1708 is configured to provide graphical data to a display for presentation to a user. The graphical data may be representative of one or more graphical user interfaces and/or any other graphical content as may serve a particular implementation.

The communication interface 1710 can include hardware, software, or both. In any event, the communication interface 1710 can provide one or more interfaces for communication (such as, for example, packet-based communication) between the computing device 1700 and one or more other computing devices or networks. As an example, and not by way of limitation, the communication interface 1710 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1710 may facilitate communications with various types of wired or wireless networks. The communication interface 1710 may also facilitate communications using various communication protocols. The communication infrastructure 1712 may also include hardware, software, or both that couples components of the computing device 1700 to each other. For example, the communication interface 1710 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer-readable medium storing instructions that, when executed by the at least one processor, cause the system to:
determine a set of candidate alignments between one or more nucleotide reads from a genomic sample with a primary contiguous sequence at a respective set of genomic regions of a reference genome;
generate a primary alignment score for a candidate alignment from the set of candidate alignments;
identify one or more allele-variant differences among the primary contiguous sequence and one or more population haplotypes corresponding to a respective genomic region for the candidate alignment;
generate one or more adjusted alignment scores from the primary alignment score based on comparing the one or more nucleotide reads with the one or more allele-variant differences; and
select, from the set of candidate alignments, a predicted read alignment of the one or more nucleotide reads with the primary contiguous sequence or with a population haplotype from the one or more population haplotypes based on the one or more adjusted alignment scores.

2. The system of claim 1, further comprising instructions that, when executed by the at least one processor, cause the system to:
generate a replacement alignment score for the candidate alignment based on the primary alignment score and the one or more adjusted alignment scores;
generate additional replacement alignment scores for additional candidate alignments of the set of candidate alignments; and
select the predicted read alignment of the one or more nucleotide reads based on comparing the replacement alignment score with one or more primary alignment scores for one or more candidate alignments with one or more primary contiguous sequences and with the additional replacement alignment scores for the additional candidate alignments of the set of candidate alignments.

3. The system of claim 1, further comprising instructions that, when executed by the at least one processor, cause the system to:
determine, for a paired-end read of the one or more nucleotide reads, that a first candidate alignment of a first mate of the paired-end read with the primary contiguous sequence is not within a threshold number of nucleobases from a second candidate alignment of a second mate of the paired-end read with the primary contiguous sequence; and
based on the first candidate alignment not being within the threshold number of nucleobases from the second candidate alignment, identify the second candidate alignment of the second mate within a predetermined search region relative to the first candidate alignment of the first mate.

4. The system of claim 1, further comprising instructions that, when executed by the at least one processor, cause the system to identify the one or more allele-variant differences by querying a haplotype data structure comprising a set of bins corresponding to a set of reference spans of nucleobases from a reference genome.

5. The system of claim 4, further comprising instructions that, when executed by the at least one processor, cause the system to:
query the haplotype data structure by identifying a reference span of the set of reference spans that includes an entire candidate alignment of the one or more nucleotide reads; and
identify the one or more allele-variant differences stored within a bin of the set of bins corresponding to the identified reference span.

6. The system of claim 5, further comprising instructions that, when executed by the at least one processor, cause the system to identify the one or more allele-variant differences stored within the bin corresponding to the identified reference span by comparing the one or more nucleotide reads with allele-variant differences stored within the bin from one or more locally distinct population haplotype sequences.

7. The system of claim 1, further comprising instructions that, when executed by the at least one processor, cause the system to:
query, for a first mate and a second mate of a paired-end read of the one or more nucleotide reads, a haplotype data structure by identifying a reference span of a set of reference spans that includes a first candidate alignment of the first mate and a second candidate alignment of the second mate;
generate, for each locally distinct population haplotype encoded by the reference span, a first adjusted alignment score for the first mate and a second adjusted alignment score for the second mate based on comparing the first mate and the second mate with the one or more allele-variant differences stored within a bin of a set of bins corresponding to the identified reference span;
sum, for each locally distinct population haplotype encoded by the reference span, the first adjusted alignment score for the first mate and the second adjusted alignment score for the second mate; and
select, from the set of candidate alignments, a first predicted alignment of the first mate and a second predicted alignment of the second mate with the primary contiguous sequence or with a locally distinct population haplotype based on a highest sum of adjusted alignment scores.

8. The system of claim 7, further comprising instructions that, when executed by the at least one processor, cause the system to:
generate a summed replacement alignment score for a subset of candidate alignments for the first mate and the second mate based on the primary alignment score and the first adjusted alignment score and the second adjusted alignment score for each locally distinct population haplotype encoded by the reference span;
generate additional summed replacement alignment scores for additional subsets of candidate alignments of the set of candidate alignments for the first mate and the second mate; and
select, from the set of candidate alignments, the first predicted alignment and the second predicted alignment based on comparing the summed replacement alignment score with one or more primary alignment scores for one or more candidate alignments with one or more primary contiguous sequences and with the additional summed replacement alignment scores for the additional subsets of candidate alignments of the set of candidate alignments.

9. The system of claim 1, further comprising instructions that, when executed by the at least one processor, cause the system to generate the one or more adjusted alignment scores without comparing nucleobases of the one or more nucleotide reads with nucleobases of the one or more population haplotypes at base positions where there are no allele-variant differences.

10. The system of claim 1, further comprising instructions that, when executed by the at least one processor, cause the system to identify the one or more allele-variant differences by comparing nucleobases within the one or more nucleotide reads with data representing one or more single nucleotide polymorphisms (SNPs) within the one or more population haplotypes corresponding to the respective genomic region.

11. A non-transitory computer-readable medium storing instructions that, when executed by at least one processor, cause a system to:
determine a set of candidate alignments between one or more nucleotide reads from a genomic sample with a primary contiguous sequence at a respective set of genomic regions of a reference genome;
generate a primary alignment score for a candidate alignment from the set of candidate alignments;
identify one or more allele-variant differences among the primary contiguous sequence and one or more population haplotypes corresponding to a respective genomic region for the candidate alignment;
generate one or more adjusted alignment scores from the primary alignment score based on comparing the one or more nucleotide reads with the one or more allele-variant differences; and
select, from the set of candidate alignments, a predicted read alignment of the one or more nucleotide reads with the primary contiguous sequence or with a population haplotype from the one or more population haplotypes based on the one or more adjusted alignment scores.

12. The non-transitory computer-readable medium of claim 11, further storing instructions that, when executed by the at least one processor, cause the system to identify the one or more allele-variant differences by comparing the one or more nucleotide reads with data representing one or more insertions or deletions (indels) within the one or more population haplotypes corresponding to the respective genomic region.

13. The non-transitory computer-readable medium of claim 11, further storing instructions that, when executed by the at least one processor, cause the system to generate at least one adjusted alignment score of the one or more adjusted alignment scores from the primary alignment score by:
determining that the one or more nucleotide reads comprise one or more haplotype nucleotide variants of a locally distinct population haplotype that differ from the primary contiguous sequence in the respective genomic region; and
increasing, based on the one or more nucleotide reads comprising the one or more haplotype nucleotide variants, the primary alignment score to generate the at least one adjusted alignment score.

14. The non-transitory computer-readable medium of claim 11, further storing instructions that, when executed by the at least one processor, cause the system to generate at least one adjusted alignment score of the one or more adjusted alignment scores from the primary alignment score by:
determining that the one or more nucleotide reads comprise one or more reference nucleobases of the primary contiguous sequence that differ from a locally distinct population haplotype in the respective genomic region; and
decreasing, based on the one or more nucleotide reads comprising one or more reference nucleobases, the primary alignment score to generate the at least one adjusted alignment score.

15. The non-transitory computer-readable medium of claim 11, further storing instructions that, when executed by the at least one processor, cause the system to:
generate the one or more adjusted alignment scores by generating a set of adjusted alignment scores for a respective set of locally distinct population haplotypes corresponding to the respective genomic region of the candidate alignment;
select, as a replacement alignment score for the candidate alignment, a highest adjusted alignment score from the set of adjusted alignment scores; and
select the predicted read alignment from the set of candidate alignments based on the replacement alignment score.

16. The non-transitory computer-readable medium of claim 11, further storing instructions that, when executed by the at least one processor, cause the system to:
generate the one or more adjusted alignment scores by generating a set of adjusted alignment scores for a respective set of locally distinct population haplotypes corresponding to the respective genomic region of the candidate alignment;
convert the set of adjusted alignment scores to a set of alignment likelihoods;
adjust the set of alignment likelihoods based on corresponding allele frequencies to generate a set of adjusted alignment likelihoods;
convert a summation of the set of adjusted alignment likelihoods to a replacement alignment score for the candidate alignment; and
select the predicted read alignment from the set of candidate alignments based on the replacement alignment score.

17. A computer-implemented method comprising:
determining a set of candidate alignments between one or more nucleotide reads from a genomic sample with a primary contiguous sequence at a respective set of genomic regions of a reference genome;
generating a primary alignment score for a candidate alignment from the set of candidate alignments;
identifying one or more allele-variant differences among the primary contiguous sequence and one or more population haplotypes corresponding to a respective genomic region for the candidate alignment;
generating one or more adjusted alignment scores from the primary alignment score based on comparing the one or more nucleotide reads with the one or more allele-variant differences; and
selecting, from the set of candidate alignments, a predicted read alignment of the one or more nucleotide reads with the primary contiguous sequence or with a population haplotype from the one or more population haplotypes based on the one or more adjusted alignment scores.

18. The computer-implemented method of claim 17, further comprising:
generating a replacement alignment score for the candidate alignment based on the primary alignment score and the one or more adjusted alignment scores;
generating additional replacement alignment scores for additional candidate alignments of the set of candidate alignments; and
selecting the predicted read alignment of the one or more nucleotide reads based on comparing the replacement alignment score with one or more primary alignment scores for one or more candidate alignments with one or more primary contiguous sequences and with the additional replacement alignment scores for the additional candidate alignments of the set of candidate alignments.

19. The computer-implemented method of claim 17, further comprising adjusting at least one of the one or more adjusted alignment scores based on a population allele frequency of a population haplotype within a sample population.

20. The computer-implemented method of claim 17, wherein generating the primary alignment score comprise generating the primary alignment score for the candidate alignment based on a given candidate alignment between the one or more nucleotide reads and a modified version of the primary contiguous sequence comprising one or more multi-base codes representing one or more single nucleotide polymorphisms (SNPs) or representing one or more insertions or deletions (indels).
